(19) United States
(12) Patent Application Publication
(10) Pub. No.: US 2015/0274657 A1
(43) Pub. Date: Oct. 1, 2015

(54) PYRROLIDINES

(71) Applicant: MERCK PATENT GMBH, DARMSTADT (DE)

(72) Inventors: Cyril Montagne, Saint Genis-Pouilly (FR); Jerome Molette, Versonnex (FR); Julie Routier, Ferney-Voltaire (FR)

(73) Assignee: MERCK PATENT GMBH, DARMSTADT (DE)

(21) Appl. No.: 14/432,351

(22) PCT Filed: Sep. 10, 2013
(86) PCT No.: PCT/EP2013/002717

§ 371 Date: Mar. 30, 2015

(30) Foreign Application Priority Data
Oct. 2, 2012 (EP) 12186958.0

(51) Int. Cl. C07D 207/16; C07D 401/12; A61K 31/4439; A61K 31/4725; A61K 31/5377; A61K 31/4025; C07D 417/14; C07D 401/06; A61K 31/454; C07D 471/04; A61K 31/437; A61K 31/4375; A61K 31/40; A61K 31/496

(52) U.S. Cl. CPC C07D 207/16; A61K 31/40; C07D 401/12; A61K 31/4439; A61K 31/4725; A61K 31/5377; A61K 31/496; C07D 417/14; C07D 401/06; A61K 31/454; C07D 471/04; A61K 31/437; A61K 31/4375; A61K 31/4025

(57) ABSTRACT

The invention relates to compounds of formula I:

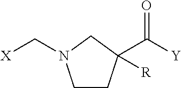

wherein:
R is H or alkyl,
X denotes one of the following groups:

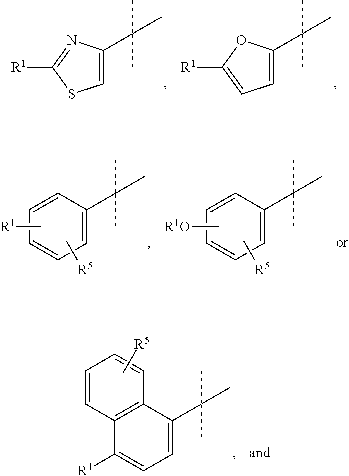

Y is OH, Oalkyl, NR²R³. The compounds are useful e.g. in the treatment of autoimmune and/or inflammatory disorders, such as multiple sclerosis.

The present invention relates to pyrrolidine derivatives, their use as medicaments and their use for treating inflammatory disorders and other diseases.

Specifically, the invention relates to the compounds of formula (I):

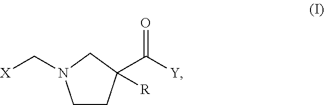

wherein
R is H or alkyl,
X denotes one of the following groups:

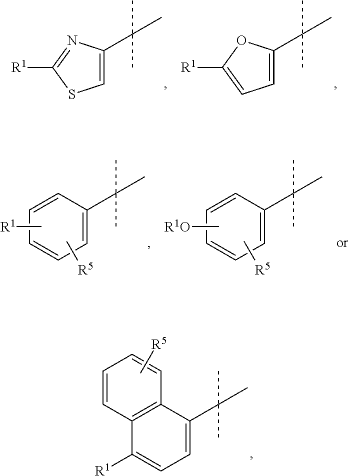

wherein
R¹ is Ar, Het or A,
Ar denotes a monocyclic or bicyclic, unsaturated or aromatic carbocyclic ring having 6 to 14 carbon atoms which may be unsubstituted or monosubstituted, disubstituted or trisubstituted by Hal, A, preferably alkyl, CH₂OR, CH₂NR₂, OR, NR₂, NO₂, CN, COOR, CF₃, OCF₃, CONR₂, COR, phenyl and/or pyridyl
Het denotes a monocyclic or bicyclic, saturated, unsaturated or aromatic heterocyclic ring having 1 to 3 N, O and/or S atoms which may be unsubstituted or monosubstituted, disubstituted or trisubstituted by Hal, A, CH₂OR, CH₂NR₂, OR, CF₃, OCF₃, NR₂, NO₂, CN, COOR, CONR₂, COR, phenyl and/or pyridyl,
Y is OH, Oalkyl, NR²R³,
   wherein
R² is H or A and
R³ is A,
A is branched or linear alkyl having 1 to 12 C-atoms, wherein one or more, such as 1 to 7, H atoms may be replaced by Ar, such as phenyl, Het, Hal, OR, CN or NR₂ and wherein one or more, preferably 1 to 3 CH₂-groups may be replaced by CO, phenylene, such as 1,4-phenylene, O, NR or S and/or by —CH═CH— or —C≡C— groups, or denotes cycloalkyl or cycloalkylalkylen having 3-7 ring C atoms or, alternatively,
NR²R³ is selected from the following group:

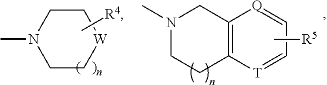

wherein
W is CHR⁶, NR⁷, O,
R⁴ is H, OH, alkyl, Oalkyl,
R⁵ is H, Hal, A, CH₂OR, CH₂NR₂, OR, NR₂, NO₂, CN, COOR, CF₃, OCF₃, CONR₂, COR, phenyl and/or pyridyl,
R⁶ is H or A,
R⁷ is H or alkyl,
Q, T is independently of one another N or CR⁸
R⁸ is H or A
n is 0, 1 or 2.
Hal denotes F, Cl, Br, I
   and pharmaceutically usable derivatives, solvates, salts and stereoisomers thereof, including mixtures thereof in all ratios.

The compounds of the present invention are particularly useful in the prophylaxis and treatment of autoimmune and/or inflammatory disorders, including neurodegenerative diseases, such as multiple sclerosis, polyneuritis, multiple neuritis, amyotrophic lateral sclerosis (ALS), Alzheimer's disease and Parkinson's disease. Preferred compound of formula I are sodium channel blockers such as Nav 1.6 inhibitors.

Mechanistics studies on white matter damage have shown that exposure of axons to hypoxia leads to excessive sodium influx and a consequent inverse functioning of the sodium-calcium exchanger (NCX) that ultimately triggers activation of calcium-mediated cell death cascades. Experimentally, this idea is supported by a large body of experimental observations including that blocking of sodium channels with Tetrodotoxin (TTX) or saxitosin, blocking of the NCX (with bepridil, benzamil, dichlorobenzamil) or manipulation of the transmembrane sodium gradient by substituting Na+ with Li+ or choline can all protect axons against anoxic injury. Conversely, increasing sodium channel permeability during anoxia with veratridine resulted in greater injury (Stys et al., 1992, Basaniak et al., 2004). Under hypoxic conditions, the availability of adenosine triphosphate (ATP) within is axoplasm becomes limited not only due to decrease synthesis but also due to the increased demands from the sodium-potassium adenosine triphosphatase (Na+/K+-ATPase) for extruding exceeding sodium. It has also been shown that in an inflammatory milieu, where nitric oxide (NO) and reactive oxygen species (ROS) are produced by phagocytic cells such as macrophages and microglia, the availability of ATP is diminished by the damage that this mediators can directly cause on mitochondria, particularly on enzymes involved in the synthesis of ATP itself. Through this mechanism, NO donors can exacerbate the axonal damage induced by hypoxia (Kappor et al., 2003). Indeed, in multiple sclerosis, where a persistent sodium current is hypothesized to overload demyelinated axons and where the synthesis of ATP is affected by NO and reactive oxygen species (ROS) due to the inflammatory nature of this disease, any initial Na+ overload cannot be overcome and creates a vicious cycle, causing reverse functioning of the NCX which in turn activates Ca+2-mediated cell cascades including the increased synthesis of NO, which besides impairing ATP synthesis itself, in addition to triggering axonal degeneration and apoptosis by multiple known mechanisms. This aspect of the pathology of multiple sclerosis is well documented in the literature and has been named virtual hypoxia (Stys, 2005; Waxman 2006; Waxman 2008).

In line with the hypothesis of the association of sodium overload and axonal degeneration in multiple sclerosis are the observations of increased total sodium content in the advanced stage of relapsing-remitting (RR) multiple sclerosis, especially in the normal-appearing brain tissues by using sodium 23 (23Na) magnetic resonance (MR) imaging (Zaaraoui et al., 2012). Sodium channel blockers such as Phenytoin, Carbamazepine, Flecainide and Lamotrigine are well established drugs and are indicated for different conditions such as epilepsy, neuropathic pain and arrhythmia. All these compound have one feature in common, i.e., they are all state-dependent sodium channel blockers, meaning that they do not affect the normal functioning of sodium channels, but do so particularly in pathological states where higher than normal neuronal firing increases the proportion of channels that are found at any time point in a conformational configuration called inactivated state. This is crucial for the safety of these drugs given that action potentials in the central and peripheral nervous systems (CNS and PNS) and axons are conducted by voltage-gated sodium channels.

All of the above mentioned examples of VGSC blockers have been tested in EAE and have in general been shown to improve clinical scores, ameliorate the axonal loss and demyelination associated with disease and revert the loss in axonal conductivity in the spinal cord of the test animals (Lo et al., 2003; Black et al., 2006; Lo et al., 2002; Craner et al., 2005; Betchold et al., 2004; Betchold et al., 2005; Betchold et al., 2006; Black et al., 2007). Voltage-gated sodium channel blockers also exhibit a protective effect in other disease models including spinal cord injury which is a relevant CNS injury model. Collectively, the body of evidence discussed above was convincing enough to raise interest within the scientific community to test the efficacy of VGSC as neuroprotective agents and Lamotrigine was tested in a randomised, double-blind phase II clinical trial for neuroprotection in secondary progressive MS patients and Lamotrigine treatment reduced the deterioration of the timed 25-foot walk (p=0.02) over 2 years.

Two voltage-gated sodium channel (VGSC) isoforms namely Nav1.2 and Nav1.6 have been shown to be overexpressed in post-morten tissue from multiple sclerosis patients and in different animal models mimicking the disease and that are collectively known as experimental autoimmune encephalomyelitis (EAE) (Craner et al., 2004a,b, Craner et al., 2005). Amongst neurons overexpressing VGSC, those overexpressing Nav1.6 are more frequently co-localized with the degeneration marker β-amyloid precursor protein (APP) than those overexpressing Nav1.2. Indeed, it has long been known that axons selectively expressing Nav1.2 are extremely resistant to anoxic injury (Waxman et al., 1990). This is likely related to the electrophysiological properties of this channel: Nav1.2 shows greater accumulation of inactivation at high frequencies of stimulation while producing smaller persistent currents in comparison with Nav1.6 (Rush et al., 2005). On the other hand, Nav1.6 produces large persistent currents that may play a role in triggering reverse functioning of the NCX which can injure demyelinated axons where Nav1.6 and the NCX are co-localized. Collectively, this evidence indicates that the Nav1.6 isoform mediates axonal degeneration in multiple sclerosis (Waxmann, 2006; Waxmann 2008, (see e.g. Banasiak K J, Burenkova O, Haddad G G. Activation of voltage-sensitive sodium channels during oxygen deprivation leads to apoptotic neuronal death. Neuroscience. 2004; 126(1):31-44., Bechtold D A, Kapoor R, Smith K J. Axonal protection using flecainide in experimental autoimmune encephalomyelitis. Ann Neurol. 2004 May; 55(5):607-16., Bechtold D A, Miller S J, Dawson A C, Sun Y, Kapoor R, Berry D, Smith K J. Axonal protection achieved in a model of multiple sclerosis using lamotrigine. J Neurol. 2006 December; 253(12):1542-51., Bechtold D A, Yue X, Evans R M, Davies M, Gregson N A, Smith K J. Axonal protection in experimental autoimmune neuritis by the sodium channel blocking agent flecainide. Brain. 2005 January; 128(Pt 1):18-28., Black J A, Liu S, Hains B C, Saab C Y, Waxman S G. Long-term protection of central axons with phenytoin in monophasic and chronic-relapsing EAE. Brain. 2006 December; 129(Pt 12):3196-208., Craner M J, Damarjian T G, Liu S, Hains B C, Lo A C, Black J A, Newcombe J, Cuzner M L, Waxman S G. Sodium channels contribute to microglia/macrophage activation and function in EAE and MS. Glia. 2005 Jan. 15; 49(2):220-9., Craner M J, Hains B C, Lo A C, Black J A, Waxman S G. Co-localization of sodium channel Nav1.6 and the sodium-calcium exchanger at sites of axonal injury in the spinal cord in EAE. Brain. 2004 February; 127(Pt 2):294-303., Craner M J, Newcombe J, Black J A, Hartle C, Cuzner M L, Waxman S G. Molecular changes in neurons in multiple sclerosis: altered axonal expression of Nav1.2 and Nav1.6 sodium channels and Na+/Ca2+ exchanger. Proc Natl Acad Sci USA. 2004 May 25; 101(21):8168-73., Kapoor R, Davies M, Blaker P A, Hall S M, Smith K J. Blockers of sodium and calcium entry protect axons from nitric oxide-mediated degeneration. Ann Neurol. 2003, February; 53(2):174-80., Kapoor R, Furby J, Hayton T, Smith K J, Altmann D R, Brenner R, Chataway J, Hughes R A, Miller D H. Lamotrigine for neuroprotection in secondary progressive multiple sclerosis: a randomised, double-blind, placebo-controlled, parallel-group trial. Lancet Neurol. 2010 July; 9(7):681-8., Lo A C, Black J A, Waxman S G. Neuroprotection of axons with phenytoin in experimental allergic encephalomyelitis. Neuroreport. 2002 Oct. 28; 13(15):1909-12., Lo A C, Saab C Y, Black J A, Waxman S G. Phenytoin protects spinal cord axons and preserves axonal conduction and neurological function in a model of neuroinflammation in vivo. J Neurophysiol. 2003 November; 90(5):3566-71., Rush A M, Dib-Hajj S D, Waxman S G. Electrophysiological properties of two axonal sodium channels, Nav1.2 and Nav1.6, expressed in mouse spinal sensory neurones. J Physiol. 2005 May 1; 564(Pt 3):803-15., Stys P K, Waxman S G, Ransom B R. Ionic mechanisms of anoxic injury in mammalian CNS white matter: role of Na+ channels and Na(+)-Ca2+ exchanger. J Neurosci. 1992 February; 12(2):430-9., Stys P K. General mechanisms of axonal damage and its prevention. J Neurol Sci. 2005 Jun. 15; 233(1-2):3-13., Waxman S G, Davis P K, Black J A, Ransom B R. Anoxic injury of mammalian central white matter: decreased susceptibility in myelin-deficient optic nerve. Ann Neurol. 1990 September; 28(3):335-40., Waxman S G. Axonal conduction and injury in multiple sclerosis: the role of sodium channels. Nat Rev Neurosci. 2006 December; 7(12):932-41., Waxman S G. Mechanisms of disease: sodium channels and neuroprotection in multiple sclerosis-current status. Nat Clin Pract Neurol. 2008 March; 4(3):159-69., Zaaraoui W, Konstandin S, Audoin B, Nagel A M, Rico A, Malikova I, Soulier E, Viout P, Confort-Gouny S, Cozzone P J, Pelletier J, Schad L R, Ranjeva J P. Distribution of Brain Sodium Accumulation Correlates with Disability in Multiple Sclerosis: A Cross-sectional 23Na MR Imaging Study. Radiology. 2012 Jul. 17. [Epub ahead of print]).

Above and below, the radicals or parameters R, R¹, R², R³, R⁴, R⁵, R⁶, R⁷, R⁸, W, Q, T, X, Y, m and n have the meaning indicated under the formula I, unless expressly stated otherwise.

Alkyl denotes unbranched (linear) or branched, and has 1, 2, 3, 4, 5, 6, 7, 8, 9, 10, 11 or 12 carbon atoms. Alkyl preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethyl-propyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl.

A preferably denotes branched or linear alkyl having 1 to 12 C-atoms, wherein 1 to 3H atoms may be replaced by Hal, OR, CN or NR₂ and/or wherein 1H atom may be replaced by Ar such as phenyl or Het and wherein one or more, preferably 1 to 3 CH₂-groups may be replaced by CO, O, NH. A very particularly preferably denotes alkyl having 1, 2, 3, 4, 5 or 6 carbon atoms, preferably methyl, ethyl, propyl, isopropyl, butyl, isobutyl, sec-butyl, tert-butyl, pentyl, hexyl, trifluoromethyl, pentafluoroethyl, 1,1,1-trifluoroethyl. In a preferred embodiment A is perfluorated alkyl. A furthermore denotes (CH₂)ₙOCH₃, especially —(CH₂)₂OCH₃, and, when bound to a carbon atom also preferably OAr, such as Ophenyl, OHet Oalkyl, such as Omethyl or Oisobutyl.

X denotes preferably one of the following groups:

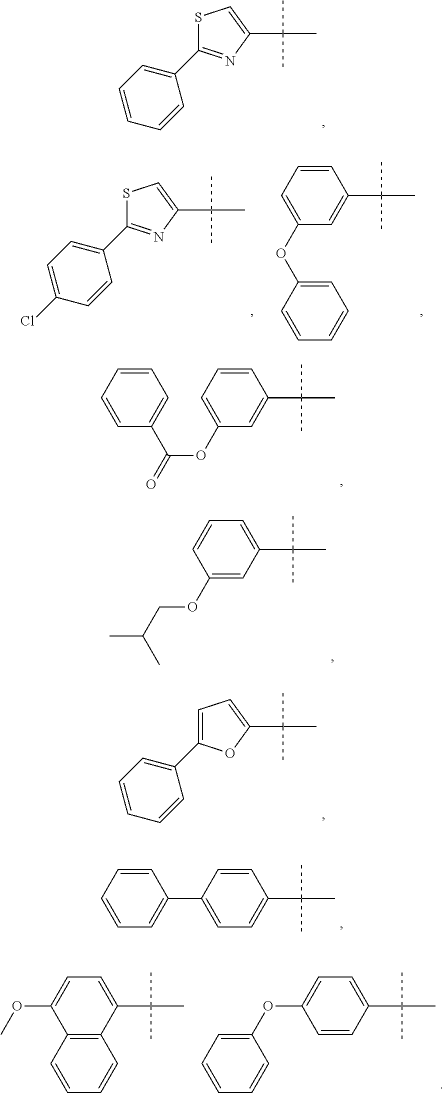

Y is preferably the group —NR²R³.

R is preferably alkyl, such as linar alkyl having 1 to 6 carbon atoms, preferably methyl or ethyl.

R¹ preferably denotes phenyl, Ophenyl, Oalkyl, such as Oisobutyl or methoxy.

R² is preferably H or alkyl such as methyl

R³ is preferably alkyl, such as ethyl, n-butyl, —(CH₂)₂OCH₃, or denotes one of the following groups:

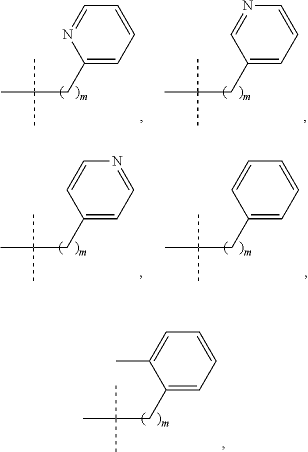

wherein m is 0, 1, 2, 3 or 2, preferably 1 or 2,
or alternatively, the group —NR²R³ preferably denotes one of the following groups:

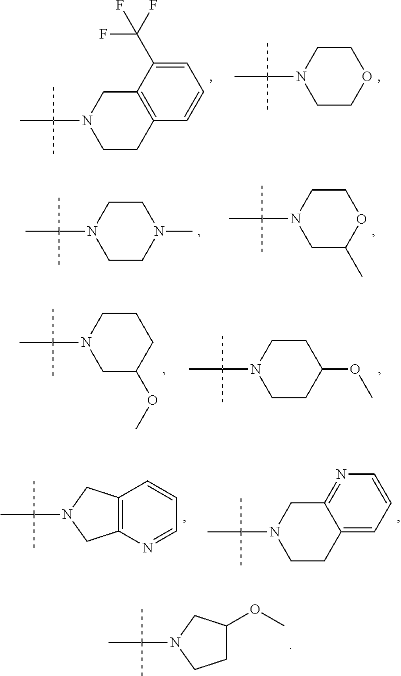

R⁴ is preferably H, alkyl, such as methyl, OH, Oalkyl, such as methoxy.

R⁵ is preferably H, OH, —OCH₃, —OCF₃, —CH₃, —NO₂, Hal, or —CF₃

R⁶ is preferably H, OH or methoxy.

R⁷ is preferably H or alkyl, such as methyl or ethyl.

R⁸ is preferably H.

Hal is preferably F, Cl or Br and especially F or Cl.

W preferably denotes O, NH or NCH₃.

Q preferably denotes N or CCF₃.

T preferably denotes CH.

n is preferably 0 or 1.

An aromatic carbocyclic ring Ar preferably denotes phenyl, naphthyl or biphenyl.

Ar denotes, for example, phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-nitrophenyl, o-, m- or p-aminophenyl, o-, m- or p-(N-methylamino)phenyl, o-, m- or p-(N-methylaminocarbonyl)-phenyl, o-, m- or p-acetamidophenyl, o-, m- or p-methoxyphenyl, o-, m- or p-ethoxyphenyl, o-, m- or p-ethoxycarbonylphenyl, o-, m- or p-(N,N-dimethyl-amino)phenyl, o-, m- or p-(N,N-dimethylaminocarbonyl)phenyl, o-, m- or p-(N-ethylamino)phenyl, o-, m- or p-(N,N-diethylamino)phenyl, o-, m- or p-fluorophenyl, o-, m- or p-bromophenyl, o-, m- or p-chlorophenyl, o-, m- or p-(methylsulfonamido)phenyl, o-, m- or p-(methylsulfonyl)phenyl, o, m or p-amino-sulfanyl-phenyl, o-, m- or p-phenoxyphenyl, further preferably 2,3-, 2,4-, 2,5-, 2,6-, 3,4- or 3,5-dimethylphenyl, 2,3-, 2,4-, 2,5-, 2,6-, 3,4- or 3,5-di-fluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-tri-methoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methyl-phenyl, 3-chloro-4-acetamidophenyl or 2,5-dimethyl-4-chlorophenyl.

Ar preferably denotes, for example, phenyl which is unsubstituted or monosubstituted, disubstituted or trisubstituted by A, Hal, OR³, CF₃, OCF₃, NO₂ and/or CN. if Ar is phenyl, it is preferably substituted in 2′position, i.e. in ortho-position to the oxadiazole bearing moiety. Ar is preferably substituted by A, OR³, CF₃ OCF₃.

Ar particularly preferably denotes, for example, phenyl which is unsubstituted or monosubstituted or disubstituted preferably monosubstituted, by F, OCH₃, CH₃, CF₃, phenyl and/or pyridyl, such as, for example, 2′-methoxy-phenyl-, 2′-trifluoromethyl-phenyl- (aryl bearing at least a 2′ substituent), 2′-chloro-phenyl, 2′,6′-dimethyl-phenyl- or 2′-alkyl-phenyl-, preferably 2′-methyl-phenyl.

Het is preferably a 6 to 14 membered ring system and denotes, not withstanding further substitutions, for example, 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, indazolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzo-thiazolyl, 2-, 4-, 5-, 6- or 7-benzisothiazolyl, 4-, 5-, 6- or 7-benz-2,1,3-oxa-diazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, furthermore preferably 1,3-benzodioxol-5-yl, 1,4-benzodioxane-6-yl, 2,1,3-benzothiadiazol-4- or -5-yl or 2,1,3-benzoxadiazol-5-yl.

The heterocyclic radicals may also be partially or fully hydrogenated. Het can thus also denote, for example, 2,3-dihydro-2-, -3-, -4- or -5-furyl, 2,5-dihydro-2-, -3-, -4- or -5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxaneyl, 1,3-dioxane-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydro-benzofuran-5- or -6-yl, 2,3-(2-oxomethylenedioxyl)phenyl or also 3,4-dihydro-2H-1,5-benzodioxepin-6- or -7-yl, furthermore preferably 2,3-dihydrobenzo-furanyl or 2,3-dihydro-2-oxofuranyl.

If Het denotes an N-Atom bearing saturated heterocycle, Het is preferably linked to the rest of the molecule via this N-Atom.

The compounds of the formula (I) can have one or more centres of chirality and can therefore occur in various stereoisomeric forms. The formula (I) includes all these forms.

Accordingly, the invention relates, in particular, to compounds of Formula (I) and its use, in which at least one of the said radicals has one of the preferred meanings indicated above.

Especially preferred are the following compounds of formula (I), which are presented with their respective activity:

| | | Nav1.6 | Nav1.6 |
|---|---|---|---|
| Example | | Inactivated | Tonic |
| Number | | (μM) | (μM) |
| 1 | 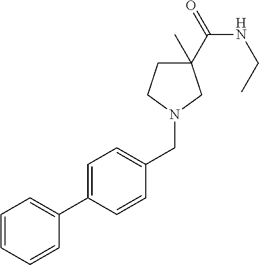 [SMILES: CCNC(=O)C1(C)CCN(Cc2ccc(-c3ccccc3)cc2)C1] | 1.77 | 21.4 |
| 2 | 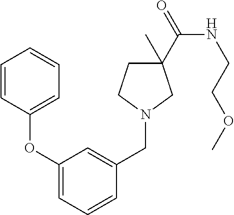 [SMILES: COCCNC(=O)C1(C)CCN(Cc2cccc(Oc3ccccc3)c2)C1] | 0.993 | 15.5 |
| 4 | 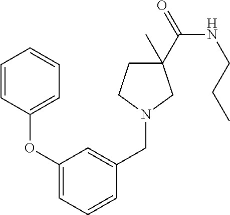 [SMILES: CCCNC(=O)C1(C)CCN(Cc2cccc(Oc3ccccc3)c2)C1] | 1.03 | 9.42 |
| 5 | 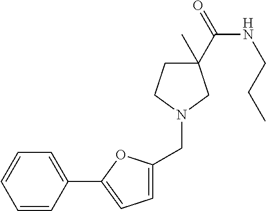 [SMILES: CCCNC(=O)C1(C)CCN(Cc2ccc(-c3ccccc3)o2)C1] | 0.785 | 30.7 |
| 6 | 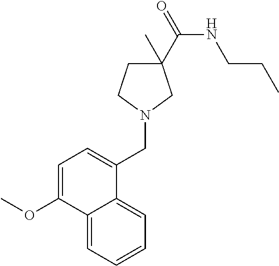 [SMILES: CCCNC(=O)C1(C)CCN(Cc2ccc(OC)c3ccccc23)C1] | 1.86 | 23.4 |
| 7 | 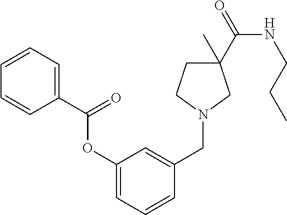 [SMILES: CCCNC(=O)C1(C)CCN(Cc2cccc(OC(=O)c3ccccc3)c2)C1] | 1.61 | >33 |
| 8 | 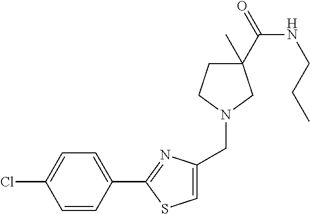 [SMILES: CCCNC(=O)C1(C)CCN(Cc2csc(-c3ccc(Cl)cc3)n2)C1] | 1.03 | 14.8 |
| 9 | 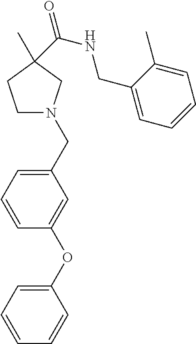 [SMILES: Cc1ccccc1CNC(=O)C1(C)CCN(Cc2cccc(Oc3ccccc3)c2)C1] | 0.615 | 14.1 |
| 10 | 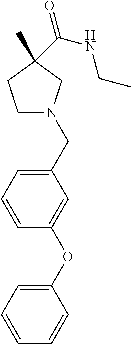 [SMILES: CCNC(=O)[C@@]1(C)CCN(Cc2cccc(Oc3ccccc3)c2)C1] | 0.808 | 20 |
| 11 | 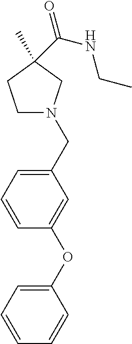 [SMILES: CCNC(=O)[C@]1(C)CCN(Cc2cccc(Oc3ccccc3)c2)C1] | 0.825 | 15.7 |
| 12 | 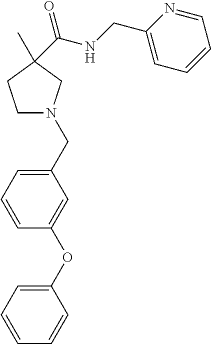 [SMILES: CC1(C(=O)NCc2ccccn2)CCN(Cc2cccc(Oc3ccccc3)c2)C1] | 0.228 | >33 |
| 13 | 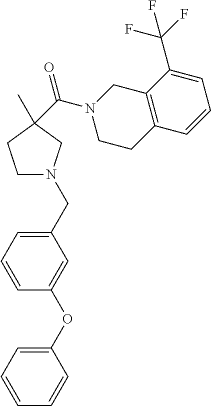 [SMILES: CC1(C(=O)N2CCc3cccc(C(F)(F)F)c3C2)CCN(Cc2cccc(Oc3ccccc3)c2)C1] | 1.97 | >33 |
| 14 | 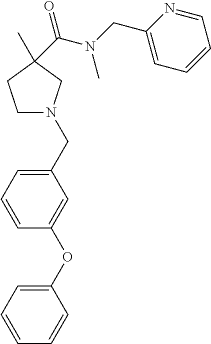 [SMILES: CN(Cc1ccccn1)C(=O)C1(C)CCN(Cc2cccc(Oc3ccccc3)c2)C1] | 0.473 | 30.3 |
| 15 | 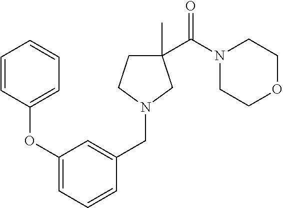 [SMILES: CC1(C(=O)N2CCOCC2)CCN(Cc2cccc(Oc3ccccc3)c2)C1] | 0.733 | 15.5 |
| 16 | 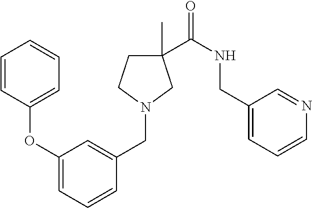 [SMILES: CC1(C(=O)NCc2cccnc2)CCN(Cc2cccc(Oc3ccccc3)c2)C1] | 0.409 | 13.1 |
| 17 | 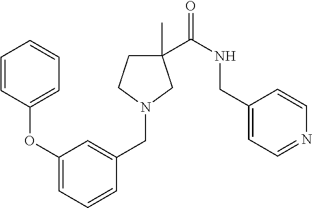 [SMILES: CC1(C(=O)NCc2ccncc2)CCN(Cc2cccc(Oc3ccccc3)c2)C1] | 0.94 | 16.4 |
| 18 | 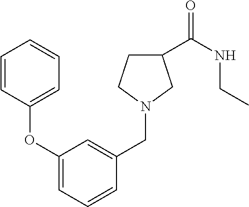 [SMILES: CCNC(=O)C1CCN(Cc2cccc(Oc3ccccc3)c2)C1] | 0.621 | 23.1 |
| 19 | 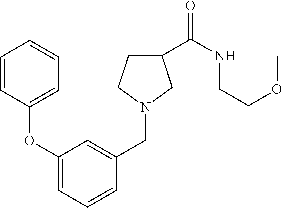 [SMILES: COCCNC(=O)C1CCN(Cc2cccc(Oc3ccccc3)c2)C1] | 1.18 | 30.3 |
| 20 | 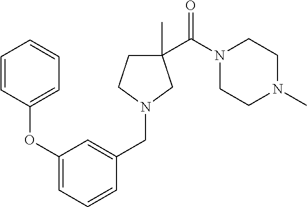 [SMILES: CN1CCN(C(=O)C2(C)CCN(Cc3cccc(Oc4ccccc4)c3)C2)CC1] | 1.91 | 8.94 |
| 21 | 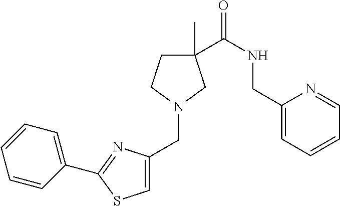 [SMILES: CC1(C(=O)NCc2ccccn2)CCN(Cc2csc(-c3ccccc3)n2)C1] | 1.08 | >33 |
| 22 | 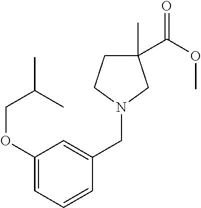 [SMILES: COC(=O)C1(C)CCN(Cc2cccc(OCC(C)C)c2)C1] | 0.994 | >33 |
| 23 | 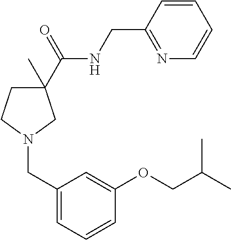 [SMILES: CC(C)COc1cccc(CN2CCC(C)(C(=O)NCc3ccccn3)C2)c1] Chiral | 0.445 | >33 |
| 24 | 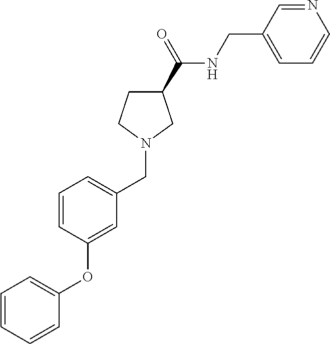 [SMILES: O=C(NCc1cccnc1)[C@@H]1CCN(Cc2cccc(Oc3ccccc3)c2)C1] | 1.3 | 29.9 |
| 25 | 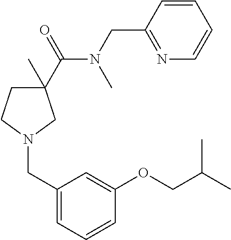 [SMILES: CC(C)COc1cccc(CN2CCC(C)(C(=O)N(C)Cc3ccccn3)C2)c1] | 0.752 | 19.9 |
| 26 | 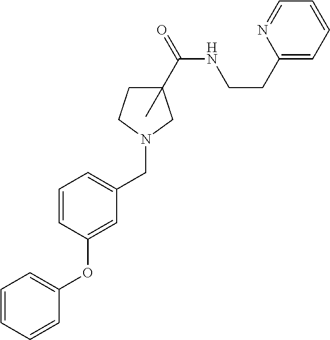 | 0.771 | 10.6 |
| 27 | 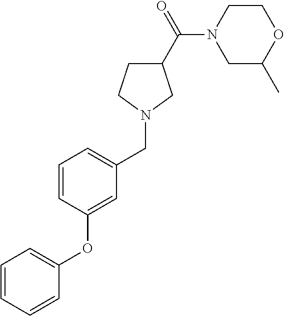 [SMILES: CC1CN(C(=O)C2CCN(Cc3cccc(Oc4ccccc4)c3)C2)CCO1] | 1.44 | >33 |
| 28 | 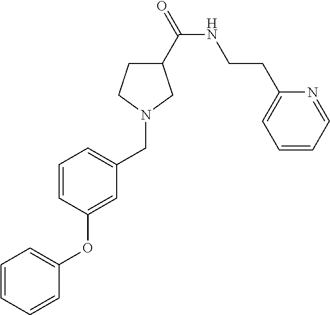 [SMILES: O=C(NCCc1ccccn1)C1CCN(Cc2cccc(Oc3ccccc3)c2)C1] | 1.17 | 19.6 |
| 29 | 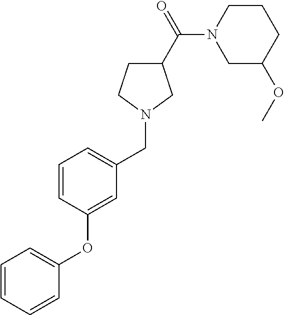 [SMILES: COC1CCCN(C(=O)C2CCN(Cc3cccc(Oc4ccccc4)c3)C2)C1] | 1.89 | >33 |
| 30 | 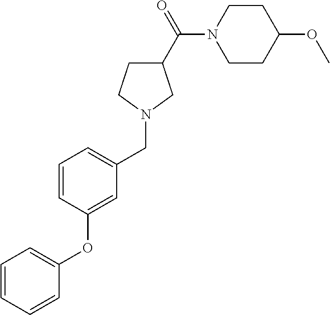 [SMILES: COC1CCN(C(=O)C2CCN(Cc3cccc(Oc4ccccc4)c3)C2)CC1] | 1.09 | >33 |
| 31 | 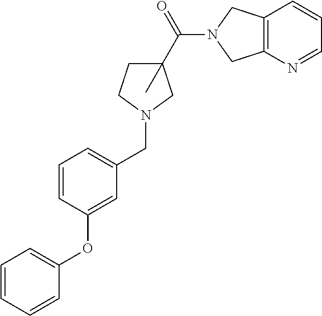 | 1.74 | >33 |
| 32 | 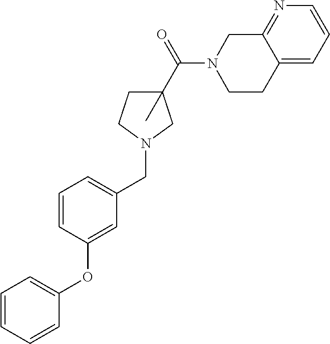 | 1.67 | >33 |
| 33 | 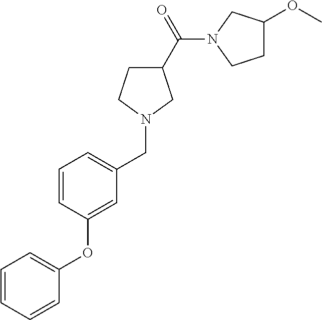 [SMILES: COC1CCN(C(=O)C2CCN(Cc3cccc(Oc4ccccc4)c3)C2)C1] | 1.52 | >33 |
| 34 | 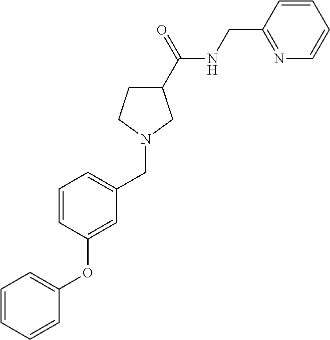 [SMILES: O=C(NCc1ccccn1)C1CCN(Cc2cccc(Oc3ccccc3)c2)C1] | 0.353 | 16.2 |

The present invention furthermore relates to a method of treating a subject suffering from an immunerogulatory abnomality, comprising administering to said subject a compounds of formula I in an amount that is effective for treating said immunoregulatory abnormality. The present invention preferably relates to a method wherein the immunoregulatory abnormality is an autoimmune or chronic inflammatory disease selected from the group consisting of: amyotrophic lateral sclerosis (ALS), Alzheimer's disease, Parkinson's disease, systemic lupus erythematosus, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves' ophthalmopathy and asthma. The present invention furthermore relates to a method wherein the immunoregulatory abnormality is bone marrow or organ transplant rejection or graft-versus-host disease. The present invention furthermore relates to a method wherein the immunoregulatory abnormality is selected from the group consisting of: transplantation of organs or tissue, graft-versus-host diseases brought about by transplantation, autoimmune syndromes including rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, post-infectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, Vernal conjunctivitis, uveitis associated with Behcet's disease, keratitis, herpetic keratitis, conical cornea, dystrophia epithelialis corneae, corneal leukoma, ocular pemphigus, 5 Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemiareperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C4 release, Behcet's disease, autoimmune hepatitis, primary biliary cirrhosis, sclerosing cholangitis, partial liver resection, 35 acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, trauma, and chronic bacterial infection.

The compounds of the formula (I) and also the starting materials for the preparation thereof are, in addition, prepared by methods known per se, as described in the literature (for example in the standard works, such as Houben-Weyl, Methoden der organischen Chemie [Methods of Organic Chemistry], Georg-Thieme-Verlag, Stuttgart), under reaction conditions which are known and suitable for the said reactions. For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3^{rd} Edition 1999.

Use can also be made here of variants which are known per se, but are not mentioned here in greater detail.

If desired, the starting materials can also be formed in situ so that they are not isolated from the reaction mixture, but instead are immediately converted further into the compounds of the formula (I).

The starting compounds for the preparation of compounds of formula (I) are generally known. If they are novel, they can, however, be prepared by methods known per se.

The reactions are preferably carried out in an inert solvent.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

### Pharmaceutical Salts and Other Forms

The said compounds of the formula I can be used in their final non-salt form. On the other hand, the present invention also relates to the use of these compounds in the form of their pharmaceutically acceptable salts, which can be derived from various organic and inorganic acids and bases by procedures known in the art. Pharmaceutically acceptable salt forms of the compounds of the formula (I) are for the most part prepared by conventional methods. If the compound of the formula I contains an acidic center, such as a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline earth metal hydroxides, such as barium hydroxide and calcium hydroxide; alkali metal alkoxides, for example sodium- or potassiumethoxide and sodium or potassiumpropoxide, alkalihydrides, such as sodium- or potassiumhydride; and various organic bases, such as piperidine, diethanolamine and N-methyl-glutamine, benzathine, choline, diethanolamine, ethylenediamine, meglumine, benethamine, diethylamine, piperazine and tromethamine. The aluminium salts of the compounds of the formula I are likewise included. In the case of certain compounds of the formula I, which contain a basic center, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoaryl-sulfonates, such as ethanesulfonate, toluenesulfonate and benzene-sulfonate, and other organic acids and corresponding salts thereof, such as acetate, trifluoro-acetate, tartrate, maleate, succinate, citrate, benzoate, salicylate, ascorbate and the like. Accordingly, pharmaceutically acceptable acid-addition salts of the compounds of the formula (I) include the following: acetate, adipate, alginate, arginate, aspartate, benzoate, benzene-sulfonate (besylate), bisulfate, bisulfate, bromide, butyrate, camphorate, camphor-sulfonate, caprylate, chloride, chlorobenzoate, citrate, cyclo-pentane-propionate, digluconate, dihydrogen-phosphate, dinitrobenzoate, dodecyl-sulfate, ethanesulfonate, fumarate, galacterate (from mucic acid), galacturonate, glucoheptanoate, gluco-nate, glutamate, glycerophosphate, hemi-succinate, hemisulfate, heptanoate, hexanoate, hippurate, hydro-chloride, hydrobromide, hydroiodide, 2-hydroxy-ethane-sulfonate, iodide, isethionate, isobutyrate, lactate, lactobionate, malate, maleate, malonate, mandelate, metaphosphate, methanesulfonate, methylbenzoate, mono-hydrogen-phosphate, 2-naphthalenesulfonate, nicotinate, nitrate, oxalate, oleate, palmo-ate, pectinate, persulfate, phenylacetate, 3-phenylpropionate, phosphate, phosphonate, phthalate, but this does not represent a restriction. Both types of salts may be formed or interconverted preferably using ion-exchange resin techniques.

Furthermore, the base salts of the compounds of the formula (I) include aluminium, ammonium, calcium, copper, iron(III), iron(II), lithium, magne-sium, manganese(III), manganese(II), potassium, sodium and zink salts, but this is not intended to represent a restriction. Of the above-mentioned salts, preference is given to ammonium; the alkali metal salts sodium and potassium, and the alkaline earth metal salts calcium and magnesium. Salts of the compounds of the formula (I) which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzyl-ethylen-ediamine (benzathine), dicyclohexylamine, diethanol-amine, diethyl-amine, 2-diethyl-amino-ethanol, 2-dimethyl-amino-ethanol, ethanolamine, ethylenediamine, N-ethylmorpholine, N-ethyl-piperidine, glucamine, glucosamine, histidine, hydrabamine, isopropyl-amine, lido-caine, lysine, meglumine (N-methyl-D-glucamine), morpholine, piperazine, piperidine, polyamine resins, procaine, purines, theobromine, triethanol-amine, triethylamine, trimethylamine, tripropyl-amine and tris(hydroxy-methyl)-methylamine (tromethamine), but this is not intended to represent a restriction.

Compounds of the formula (I) of the present invention which contain basic nitrogen-containing groups can be quaternised using agents such as (C1-C4)-alkyl halides, for example methyl, ethyl, isopropyl and tert-butyl chloride, bromide and iodide; di(C1-C4)alkyl sulfates, for example dimethyl, diethyl and diamyl sulfate; (C10-C18)alkyl halides, for example decyl, do-decyl, lauryl, myristyl and stearyl chloride, bromide and iodide; and aryl-(C1-C4)alkyl halides, for example benzyl chloride and phenethyl bromide. Both water- and oil-soluble compounds of the formula (I) can be prepared using such salts.

The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, me-glumine, nitrate, oleate, phosphonate, pivalate, sodium phosphate, stea-rate, sulfate, sulfosalicylate, tartrate, thiomalate, tosylate and tro-meth-amine, but this is not intended to represent a restriction.

The acid-addition salts of basic compounds of the formula (I) are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts other-wise correspond to the respective free base forms thereof.

As mentioned, the pharmaceutically acceptable base-addition salts of the compounds of the formula (I) are formed with metals or amines, such as alkali metals and alkaline earth metals or organic amines. Preferred metals are sodium, potassium, magnesium and calcium. Preferred organic amines are N,N′-dibenzylethylenediamine, chloroprocaine, choline, diethanol-amine, ethylenediamine, N-methyl-D-glucamine and procaine.

The base-addition salts of acidic compounds of the formula (I) are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts other-wise correspond to the respective free acid forms thereof.

If a compound of the formula I contains more than one group which is capable of forming pharmaceutically acceptable salts of this type, the formula (I) also encompasses multiple salts. Typical multiple salt forms include, for example, bitartrate, diacetate, difumarate, dimeglumine, di-phosphate, disodium and trihydrochloride, but this is not intended to represent a restriction.

With regard to that stated above, it can be seen that the term “pharmaceutically acceptable salt” in the present connection is taken to mean an active ingredient which comprises a compound of the formula I in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

Owing to their molecular structure, the compounds of the formula (I) can be chiral and can accordingly occur in various enantiomeric forms. They can therefore exist in racemic or in optically active form.

Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitable N-protected amino acids (for example N-benzoylproline or N-benzenesulfonylproline), or the various optically active camphorsulfonic acids. Also advantageous is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or alcoholic solvent mixtures, such as, for example, hexane/isopropanol/acetonitrile, for example in the ratio 82:15:3.

The invention furthermore relates to the use of compounds of formula (I), in combination with at least one further medicament active ingredient, preferably medicaments used in the treatment of multiple sclerosis such as cladribine or another co-agent, such as interferon, e.g. pegylated or non-pegylated interferons, preferably interferon beta and/or with compounds improving vascular function. These further medicaments, such as interferon beta, may be administered concomitantly or sequentially, e.g. by subcutaneous, intramuscular or oral routes.

These compositions can be used as medicaments in human and veterinary medicine.

Pharmaceutical formulations can be administered in the form of dosage units, which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process, which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medica-ment after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinyl-pyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The active ingredients can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compounds. Syrups can be prepared by dissolving the compounds in an aqueous solution with a suitable flavour, while elixirs are prepared using a non-toxic alcoholic vehicle. Suspensions can be for-mulated by dispersion of the compounds in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds of the formula (I) and salts, solvates and physiologically functional derivatives thereof and the other active ingredients can also be administered in the form of liposome delivery systems, such as, for exam-ple, small unilamellar vesicles, large unilamellar vesicles and multilamellar vesicles. Liposomes can be formed from various phospholipids, such as, for example, cholesterol, stearylamine or phosphatidylcholines.

The compounds of the formula I and the salts, solvates and physiologically functional derivatives thereof and the other active ingredients can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropyl-methacrylamidophenol, polyhydroxyethylaspartamidophenol or polyethylene oxide polylysine, substituted by palrnitoyl radicals. The compounds may furthermore be coupled to a class of biodegradable polymers which are suitable for achieving controlled release of a medicament, for example polylactic acid, poly-epsilon-caprolactone, polyhydroxybutyric acid, poly-orthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or sus-pended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insuf-flators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary.

Injection solutions and suspensions prepared in accordance with the rec-ipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the formula I and of the other active ingredient depends on a number of factors, including, for example, the age and weight of the animal, the precise disease condition which requires treatment, and its severity, the nature of the formulation and the method of administration, and is ultimately determined by the treating doctor or vet. However, an effective amount of a compound is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound per se.

The present invention furthermore relates to a method for treating a subject suffering from a Naᵥ associated disorder, comprising administering to said subject an effective amount of a compound of formula (I). The present invention preferably relates to a method, wherein the Naᵥ associated disorder is an autoimmune disorder or condition associated with an overactive immune response.

The present invention furthermore relates to a method of treating a subject suffering from an immunerogulatory abnomality, comprising administering to said subject a compound of formula I in an amount that is effective for treating said immunoregulatory abnormality. The present invention preferably relates to a method wherein the immunoregulatory abnormality is an autoimmune or chronic inflammatory disease.

The present invention furthermore relates to a method of providing Neuroprotection by administration of compounds of formula I to a subject in need thereof.

The pharmaceutically acceptable cationic salts of compounds of the present invention are readily prepared by reacting the acid forms with an appropriate base, usually one equivalent, in a co-solvent. Typical bases are sodium hydroxide, sodium methoxide, sodium ethoxide, sodium hydride, potassium hydroxide, potassium methoxide, magnesium hydroxide, calcium hydroxide, benzathine, choline, diethanolamine, ethylenediamine, meglumine, benethamine, diethylamine, piperazine and tromethamine. The salt is isolated by concentration to dryness or by addition of a non-solvent. In some cases, salts can be prepared by mixing a solution of the acid with a solution of the cation (sodium ethylhexanoate, magnesium oleate), employing a solvent in which the desired cationic salt precipitates, or can be otherwise isolated by concentration and addition of a non-solvent.

According to a further general process, compounds of Formula (I) can be converted to alternative compounds of Formula (I), employing suitable interconversion techniques well known by a person skilled in the art.

In general, the synthesis pathways for any individual compound of Formula (I) will depend on the specific substitutents of each molecule and upon the ready availability of intermediates necessary; again such factors being appreciated by those of ordinary skill in the art. For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley lnterscience, 3^{rd} Edition 1999.

Compounds of this invention can be isolated in association with solvent molecules by crystallization from evaporation of an appropriate solvent. The pharmaceutically acceptable acid addition salts of the compounds of Formula (I), which contain a basic center, may be prepared in a conventional manner. For example, a solution of the free base may be treated with a suitable acid, either neat or in a suitable solution, and the resulting salt isolated either by filtration or by evaporation under vacuum of the reaction solvent. Pharmaceutically acceptable base addition salts may be obtained in an analogous manner by treating a solution of compound of Formula (I) and, which contain an acid center, with a suitable base. Both types of salts may be formed or interconverted using ion-exchange resin techniques.

Depending on the conditions used, the reaction times are generally between a few minutes and 14 days, and the reaction temperature is between about −30° C. and 140° C., normally between −10° C. and 90° C., in particular between about 0° C. and about 70° C.

Compounds of the formula I can furthermore be obtained by liberating compounds of the formula I from one of their functional derivatives by treatment with a solvolysing or hydrogenolysing agent.

Preferred starting materials for the solvolysis or hydrogenolysis are those which conform to the formula (I), but contain corresponding protected amino and/or hydroxyl groups instead of one or more free amino and/or hydroxyl groups, preferably those which carry an amino-protecting group instead of an H atom bonded to an N atom, in particular those which carry an R′—N group, in which R′ denotes an amino-protecting group, instead of an HN group, and/or those which carry a hydroxyl-protecting group instead of the H atom of a hydroxyl group, for example those which conform to the formula I, but carry a —COOR″ group, in which R″ denotes a protecting group, instead of a —COOH group.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size are furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or hetero-cyclic carboxylic acids or sulfonic acids, and, in particular, alkoxy-carbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as POA; alkoxycarbonyl, such as methoxy-carbonyl, ethoxycarbonyl, 2,2,2-trichloroethoxycarbonyl, BOC (tert-butoxy-carbonyl) and 2-iodoethoxycarbonyl; aralkoxycarbonyl, such as CBZ (“carbo-benz-oxy”), 4-methoxybenzyloxycarbonyl and FMOC; and aryl-sulfonyl, such as Mtr. Preferred amino-protecting groups are BOC and Mtr, further-more CBZ, Fmoc, benzyl and acetyl.

The term “hydroxyl-protecting group” is likewise known in general terms and relates to groups which are suitable for protecting a hydroxyl group against chemical reactions, but are easy to remove after the desired chemical reac-tion has been carried out elsewhere in the molecule. Typical of such groups are the above-mentioned unsubstituted or substituted aryl, aralkyl or acyl groups, furthermore also alkyl groups. The nature and size of the hydroxyl-protecting groups are not crucial since they are removed again after the desired chemical reaction or reaction sequence; preference is given to groups having 1-20, in particular 1-10, carbon atoms. Examples of hydroxyl-protecting groups are, inter alis, benzyl, 4-methoxybenzyl, p-nitro-benzoyl, p-toluenesulfonyl, tert-butyl and acetyl, where benzyl and tert-butyl are particu-larly preferred.

The compounds of the formula (I) are liberated from their functional derivatives—depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50° C., preferably between 15 and 30° C. (room temperature).

The BOC, OtBu and Mtr groups can, for example, preferably be cleaved off using TFA in dichloromethane or using approximately 3 to 5N HCl in dioxane at 15-30° C., and the FMOC group can be cleaved off using an approximately 5 to 50% solution of dimethylamine, diethylamine or piperidine in DMF at 15-30° C.

Protecting groups which can be removed hydrogenolytically (for example CBZ, benzyl or the liberation of the amidino group from the oxadiazole derivative thereof) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° C. and pressures between about 1 and 200 bar, preferably at 20-30° C. and 1-10 bar. Hydrogenolysis of the CBZ group succeeds well, for example, on 5 to 10% Pd/C in methanol or using ammonium formate (instead of hydrogen) on Pd/C in methanol/DMF at 20-30° C.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, tri-fluoro-methylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone (NMP) or dimethyl-formamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Esters can be saponified, for example, using acetic acid or using LiOH, NaOH or KOH in water, water/THF, water/THF/ethanol or water/dioxane, at temperatures between 0 and 100° C.

Free amino groups can furthermore be acylated in a conventional manner using an acid chloride or anhydride or alkylated using an unsubstituted or substituted alkyl halide or reacted with CH₃—C(═NH)-OEt, advantageously in an inert solvent, such as dichloromethane or THF and/or in the presence of a base, such as triethylamine or pyridine, at temperatures between −60° C. and +30° C.

The following abbreviations refer to the abbreviations used below: Ac (acetyl), aq. (aqueous), DABCO (1,4-diazabicyclo[2.2.2]octane), DCE (dichloroethane), DCM (dichioromethane), DEA (dimethylamine), DIEA (diisopropylethylamine), DMF (dimethylformamide), DMP (Dess-Martin periodinane), DMSO (dimethylsulfoxide), EA (ethyl acetate), EDC (1-ethyl-3-(3-dimethylaminopropyl)carbodiimide hydrochloride), eq. (equivalent), Et (ethyl), HATU (2-(1H-7-azabenzotriazol-1-yl)-1,1,3,3-tetramethyl uranium hexafluorophosphate methanaminium), HPLC (high performance liquid chromatography), IBX (2-iodoxybenzoic acid), LC (liquid chromatography), M (molar), Me (methyl), MS (mass spectrum), NMM (N-methylmorpholine), NMO (N-methylmorpholine oxide), NMR (nuclear magnetic resonance), PDC (pyridiniumdichromate), PyBOP (benzotriazole-1-yl-oxy-tris-(dimethylamino)-phosphonium hexafluorophosphate), Rt (retention time), SFC (supercritical fluid chromatography), T3P (2,4,6-tripropyl-[1,3,5,2,4,6]trioxatriphosphinane 2,4,6-trioxide), TEA (triethylamine), THF (tetrahydrofurane), TPAP (tetrapropylammonium perruthenate).

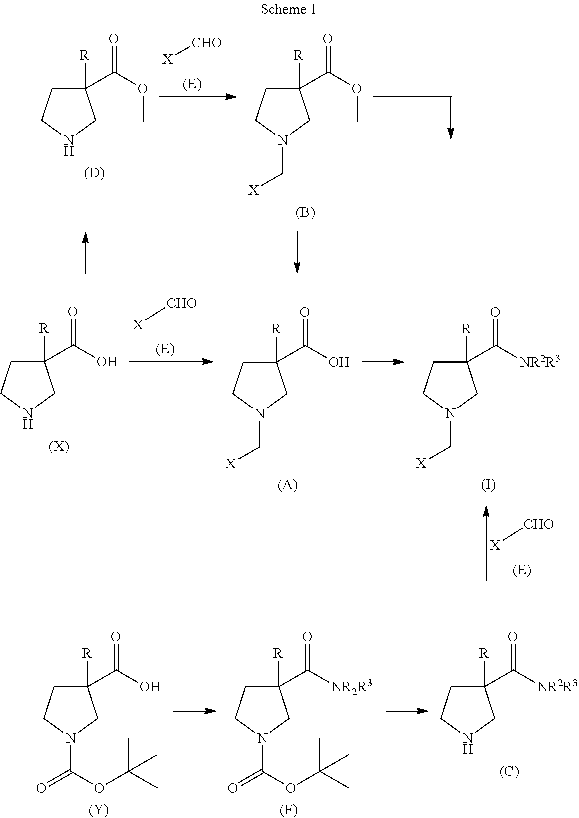

Compounds of Formula (I) wherein R, X and are as above defined and Y is NR²R³ may be prepared by reaction between a carboxylic acid of Formula (A) wherein R and X are as above defined and a primary or secondary amine of formula HNR²R³ (or a salt thereof) using coupling agents such as EDC, HATU, PyBOP, T3P in the presence or the absence of a base such as TEA, DIEA or NMM in a solvent such as DCM, DCE, THF, DMF at a temperature ranging from 0° C. and 50° C. for few minutes to several hours.

The method for preparing the compounds of Formula (I) is preferably use for the compounds selected below:
(±) 3-methyl-1-(3-phenoxy-benzyl)-pyrrolidine-3-carboxylic acid 2-methyl-benzylamide
(S)-3-methyl-1-(3-phenoxy-benzyl)-pyrrolidine-3-carboxylic acid ethylamide
(R)-3-methyl-1-(3-phenoxy-benzyl)-pyrrolidine-3-carboxylic acid ethylamide
(±) 3-methyl-1-(3-phenoxy-benzyl)-pyrrolidine-3-carboxylic acid (pyridin-2-ylmethyl)-amide
(±) [3-methyl-1-(3-phenoxy-benzyl)-pyrrolidin-3-yl]-(8-trifluoromethyl-3,4-dihydro-1H-isoquinolin-2-yl)-methanone
(±) 3-methyl-1-(3-phenoxy-benzyl)-pyrrolidine-3-carboxylic acid methyl-pyridin-2-ylmethyl-amide
(±) [3-methyl-1-(3-phenoxy-benzyl)-pyrrolidin-3-yl]-morpholin-4-yl-methanone
(±) 3-methyl-1-(3-phenoxy-benzyl)-pyrrolidine-3-carboxylic acid (pyridin-3-ylmethyl)-amide
(±) 3-methyl-1-(3-phenoxy-benzyl)-pyrrolidine-3-carboxylic acid (pyridin-4-ylmethyl)-amide
(±) 1-(3-phenoxy-benzyl)-pyrrolidine-3-carboxylic acid ethylamide
(±) 1-(3-phenoxy-benzyl)-pyrrolidine-3-carboxylic acid (2-methoxy-ethyl)-amide
(±) [3-methyl-1-(3-phenoxy-benzyl)-pyrrolidin-3-yl]-(4-methyl-piperazin-1-yl)-methanone
(±) 3-methyl-1-(2-phenyl-thiazol-4-ylmethyl)-pyrrolidine-3-carboxylic acid (pyridin-2-ylmethyl)-amide
(±) (3-methoxy-piperidin-1-yl)-[-(3-phenoxy-benzyl)-pyrrolidin-3-yl]-methanone

Alternatively, compounds of Formula (I) wherein R, and X are as above defined and Y is NR²R³ may be prepared by reaction between an ester of Formula (B) wherein R and X are as above defined and a primary or secondary amine of Formula HNR²R³ (or a salt thereof) using. AlMe₃, AlCl₃ or AlMe₃-DABCO complex in solvents such as DCM, DCE, THF or 1,4-dioxane at a temperature ranging from 0° C. to 100° C. for few minutes to several hours.

The method is preferably used for preparing the compounds of Formula (I) selected below:
(±) 1-(3-isobutoxy-benzyl)-3-methyl-pyrrolidine-3-carboxylic acid (pyridin-2-ylmethyl)-amide
(±) 1-(3-isobutoxy-benzyl)-3-methyl-pyrrolidine-3-carboxylic acid methyl-pyridin-2-ylmethyl-amide
(±) 3-methyl-1-(3-phenoxy-benzyl)-pyrrolidine-3-carboxylic acid (2-pyridin-2-yl-ethyl)-amide
(±) (2-methyl-morpholin-4-yl)-[1-(3-phenoxy-benzyl)-pyrrolidin-3-yl]-methanone
(±) 1-(3-phenoxy-benzyl)-pyrrolidine-3-carboxylic acid (2-pyridin-2-yl-ethyl)-amide
(±) (4-methoxy-piperidin-1-yl)-[1-(3-phenoxy-benzyl)-pyrrolidin-3-yl]-methanone
(±) (5,7-dihydro-pyrrolo[3,4-b]pyridin-6-yl)-[3-methyl-1-(3-phenoxy-benzyl)-pyrrolidin-3-yl]-methanone
(±) (5,8-dihydro-6H-[1,7]naphthyridin-7-yl)-[3-methyl-1-(3-phenoxy-benzyl)-pyrrolidin-3-yl]-methanone
(±) (3-methoxy-pyrrolidin-1-yl)-[1-(3-phenoxy-benzyl)-pyrrolidin-3-yl]-methanone
(±) 1-(3-phenoxy-benzyl)-pyrrolidine-3-carboxylic acid (pyridin-2-ylmethyl)-amide

Alternatively, compounds of Formula (I) wherein R, and X are as above defined and Y1 is NR²R³ may be prepared by reaction between an amine of Formula (C) wherein R, R² and R² are as above defined and an aldehyde of Formula (E) wherein X is as above defined using reducing agents such as NaBH₃CN or NaBH(OAc)₃ in the presence or the absence of an acid such acetic acid in a solvent such as DCM, DCE, THF or 1,4-dioxane at a temperature ranging from 0° C. to 100° C. for few minutes to several hours.

The method is preferably used for preparing the compounds of Formula (I) selected below:
(R)-1-(3-phenoxy-benzyl)-pyrrolidine-3-carboxylic acid (pyridin-3-ylmethyl)-amide

Alternatively, compounds of Formula (I) wherein R and X are as above defined and Y is OMe and compounds of Formula (B) wherein R and X are as above defined may be prepared by reaction between an amine of Formula (D) wherein R is as above defined and an aldehyde of Formula (E) wherein X is as above defined using reducing agents such as such as NaBH₃CN or NaBH(OAc)₃ in the presence or the absence of an acid such acetic acid in a solvent such as DCM, DCE, THF or 1,4-dioxane at a temperature ranging from 0° C. to 100° C. for few minutes to several hours.

The method is preferably used for preparing the compounds of Formula (I) selected below:
(±) 1-(3-isobutoxy-benzyl)-3-methyl-pyrrolidine-3-carboxylic acid methyl ester

Compounds of Formula (A) wherein R and X are as above defined may be prepared by reaction between an amine of Formula (X) wherein R is as above defined and an aldehyde of Formula (E) wherein X is as above defined using reducing agents such as NaBH₃CN or NaBH(OAc)₃ in the presence or the absence of an acid such acetic acid in a solvent such as DCM, DCE, THF or 1,4-dioxane at a temperature ranging from 0° C. to 100° C. for few minutes to several hours.

Alternatively compounds of Formula (A) wherein R and X are as above defined may be prepared by saponification of compounds of Formula (B) wherein R and X are as above defined using reagents such as LiOH, NaOH or KOH in a solvent such as water, THF, 1,4-dioxane, MeOH, EtOH, or a mixture thereof, at a temperature ranging from 0° C. to 100° C. for few minutes to several hours.

Compounds of Formula (C) wherein R, R² and R³ are as above defined may be prepared by deprotecting compounds of Formula (F) wherein R, R² and R³ are as above defined by reaction with an acid such as HCl or TFA in a solvent such as water, AcOH, DCM, DCE, THF or 1,4-dioxane, or a mixture thereof, at a temperature ranging from 0° C. to 100° C. for a few minutes to several hours.

Compounds of Formula (D) wherein R is as above defined may be prepared starting from a carboxylic acid of Formula (X), wherein R is as defined above, by reaction with an acid such as HCl or H₂SO₄ in a solvent such as MeOH at a temperature ranging from 0° C. to 65° C. for few minutes to several hours.

Compounds of Formula (F) wherein R, R² and R³ are above defined may be prepared by reaction between a carboxylic acid of Formula (Y) wherein R is as above defined and a primary or secondary amine of formula HNR²R³ (or a salt thereof) using coupling agents such as EDC, HATU, PyBOP, T3P in the presence or the absence of a base such as TEA, DIEA or NMM in a solvent such as DCM, DCE, THF, DMF at a temperature ranging from 0° C. and 50° C. for few minutes to several hours.

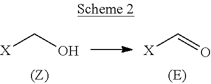

Compounds of Formula (E) wherein X is as above defined are commercially available or may be prepared by oxidation of compounds of Formula (Z) wherein X is as above defined using the Swern reaction or reagents such as MnO₂, PDC, DMP, IBX, TPAP/NMO. The reaction is preferably performed using MnO₂ in a solvent such as DCM or DCE at a temperature ranging from 0° C. to 50° C. for several hours.

In the following the present invention shall be illustrated by means of some examples, which are not construed to be viewed as limiting the scope of the invention.

### EXAMPLES

The compounds of invention have been named according to the standards used in the program AutoNom (v1.0.1.1).

The compounds according to formula (I) can be prepared from readily available starting materials by several synthetic approaches, using both solution-phase and solid-phase chemistry protocols or mixed solution and solid phase protocols. Examples of synthetic pathways are described below in the examples.

The commercially available starting materials used in the following experimental description were purchased from Aldrich, Sigma, ACROS or ABCR unless otherwise reported.

¹H NMR analyses were carried out using BRUKER NMR, model DPX-300 MHz or 400 Mhz FT-NMR. Residual signal of deuterated solvent was used as internal reference. Chemical shifts (δ) are reported in ppm in relative to the residual solvent signal (δ=2.50 for ¹H NMR in DMSO-d₆, and 7.26 in CDCl₃). s (singlet), d (doublet), t (triplet), q (quadruplet), br (broad), m (multiplet).

The MS data provided in the examples described below were obtained as followed: Mass spectrum: LC/MS Waters ZMD (ESI)

HPLC analyses were obtained as followed using a Waters Xbridge™ C8 50 mm×4.6 mm column at a flow of 2 mL/min; 8 min gradient H₂O:CH₃CN:TFA from 100:0:0.1% to 0:100:0.05% with UV detection (maxplot).

The mass directed preparative HPLC purifications were performed with a mass directed autopurification Fractionlynx from Waters equipped with a Sunfire Prep C18 OBD column 19×100 mm 5 μm, unless otherwise reported. All purifications were performed with a gradient of ACN/H₂O or ACN/H₂O/HCOOH (0.1%).

The microwave chemistry was performed on a single mode microwave reactor Emrys™ Optimiser or Initiator™ Sixty from Biotage.

### Intermediate A1: (±) 3-methyl-1-(3-phenoxy-benzyl)-pyrrolidine-3-carboxylic acid

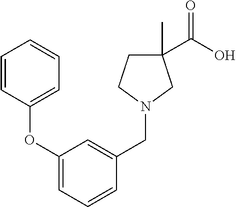 [SMILES: CC1(C(=O)O)CCN(Cc2cccc(Oc3ccccc3)c2)C1]

Sodium triacetoxyborohydride (903 mg; 4.26 mmol; 1.1 eq.) was added to a solution of 3-methyl-pyrrolidine-3-carboxylic acid (500 mg; 3.87 mmol; 1 eq.) and 3-phenoxy-benzaldehyde (767 mg; 3.87 mmol; 1 eq.) in DCM (10 mL) and the resulting mixture was stirred at room temperature for 3 hours whereupon sodium triacetoxyborohydride (903 mg; 4.26 mmol; 1.1 eq.) was added. The reaction mixture was stirred at room temperature for 16 hours. The reaction was then filtrated and the remaining solvent was evaporated under reduced pressure. Purification by mass directed preparative HPLC afforded the title compound (690 mg, 57%) as white powder. ¹H NMR (300 MHz, DMSO-d₆) δ=7.43-7.35 (m, 2H), 7.32 (t, J=7.8 Hz, 1H), 7.13 (tt, J=7.8, 0.9 Hz, 1H), 7.09-6.84 (m, 5H), 3.54 (s, 2H), 2.81 (d, J=9.0 Hz, 1H), 2.62-2.44 (m, 2H), 2.31-2.17 (m, 2H), 2.07 (s, 2H), 1.60-1.43 (m, 1H), 1.21 (s, 3H). HPLC (max plot) 99% Rt 2.86 min. LC/MS: (MS+) 312.4 (M+H⁺).

### Intermediate A2: (±) 3-methyl-1-(2-phenyl-thiazol-4-ylmethyl)-pyrrolidine-3-carboxylic acid

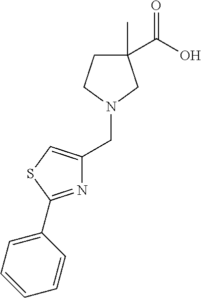 [SMILES: CC1(C(=O)O)CCN(Cc2csc(-c3ccccc3)n2)C1]

Sodium triacetoxyborohydride (496 mg; 2.34 mmol; 1.5 eq.) was added to a solution of 2-phenyl-thiazole-4-carbaldehyde (295 mg; 1.56 mmol; 1 eq.) and 3-methyl-pyrrolidine-3-carboxylic acid (211 mg; 1.64 mmol; 1.05 eq.) in DCE (15.00 mL) and the resulting mixture was stirred at room temperature for 1 hour. Water and DCM were added and the pH was adjusted to 4. The two phases were separated and the organic layer extracted with DCM (3×). The combined organics were dried over magnesium sulfate and concentrated in vacuo to afford the title compound (170 mg, 36%) as a white solid. ¹H NMR (300 MHz, DMSO-d₆) δ=8.08-7.80 (m, 2H), 7.50-7.48 (m, 4H), 3.76 (s, 2H), 3.01 (d, J=9.2 Hz, 1H), 2.68 (q, J=7.0, 6.5 Hz, 2H), 2.43 (d, J=9.2 Hz, 1H), 2.35-2.17 (m, 1H), 1.68-1.43 (m, 1H), 1.25 (s, 3H). HPLC (max plot) 92% Rt 2.43 min. LC/MS: (MS-F) 303.1 (M+H⁺).

### Intermediate A3: (±) 1-(3-phenoxy-benzyl)-pyrrolidine-3-carboxylic acid sodium salt

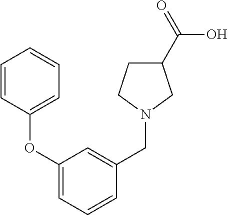 [SMILES: O=C(O)C1CCN(Cc2cccc(Oc3ccccc3)c2)C1]

A solution of Intermediate B3 (400 mg; 1.28 mmol; 1 eq.) and 5M NaOH (2.6 ml; 12.85 mmol; 10 eq.) in EtOH (5 mL) was stirred at room temperature for 4 hours then concentrated in vacuo. Purification by mass directed preparative HPLC afforded the title compound (300 mg, 79%) as white solid. HPLC (max plot) 92% Rt 2.78 min.

### Intermediate B1: (±) 1-(3-isobutoxy-benzyl)-3-methyl-pyrrolidine-3-carboxylic acid methyl ester

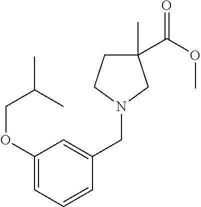 [SMILES: COC(=O)C1(C)CCN(Cc2cccc(OCC(C)C)c2)C1]

Sodium triacetoxyborohydride (307 mg; 1.45 mmol; 1.3 eq.) was added to a suspension of Intermediate D1 (200 mg; 1.11 mmol; 1 eq.) and 3-isobutoxy-benzaldehyde (218 mg; 1.22 mmol; 1.1 eq.) in DCE (2 mL) and the resulting mixture was stirred at 65° C. for 30 min then partitioned between DCM and 0.1M NaOH. The two phases were separated and the aqueous layer extracted with DCM. The combined organics were dried over sodium sulfate and concentrated in vacuo. Purification by column chromatography (10% to 20% EA in heptane) afforded the title compound (175 mg, 51%) as colourless liquid. ¹H NMR (300 MHz, DMSO-d₆) δ=7.24-7.14 (m, 1H), 6.88-6.70 (m, 3H), 3.71 (d, J=6.5 Hz, 2H), 3.61 (s, 3H), 3.51 (d, J=3.0 Hz, 2H), 2.86 (d, J=9.2 Hz, 1H), 2.63-2.51 (m, 2H), 2.33-2.22 (m, 2H), 2.08-1.90 (m, 1H), 1.68-1.50 (m, 1H), 1.26 (s, 3H), 0.97 (d, J=6.7 Hz, 6H). HPLC (max plot) 100% Rt 3.26 min. LC/MS: (MS+) 306.4 (M+H⁺).

### Intermediate B2: (±) 3-methyl-1-(3-phenoxy-benzyl)-pyrrolidine-3-carboxylic acid methyl ester

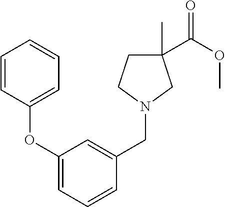 [SMILES: COC(=O)C1(C)CCN(Cc2cccc(Oc3ccccc3)c2)C1]

Sodium triacetoxyborohydride (1.65 g; 7.8 mmol; 1.4 eq.) was added to a suspension of Intermediate D1 (1 g; 5.6 mmol; 1 eq.) and 3-phenoxy-benzaldehyde (1.2 g; 6.1 mmol; 1.1 eq.) in DCE (40 mL) and the resulting mixture was stirred at 65° C. for 3 hours then partitioned between DCM and 0.1M NaOH. The two phases were separated and the aqueous layer extracted with DCM. The combined organics were dried over sodium sulfate and concentrated in vacuo. Purification by column chromatography (15% to 25% EA in heptane) afforded the title compound (1.2 g, 66%) as yellow oil. HPLC (max plot) 88% Rt 3.18 min. LC/MS: (MS+) 326.2 (M+H⁺).

### Intermediate B3: (±) 1-(3-phenoxy-benzyl)-pyrrolidine-3-carboxylic acid methyl ester

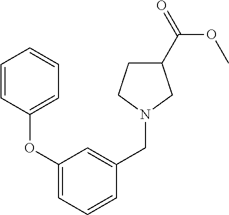 [SMILES: COC(=O)C1CCN(Cc2cccc(Oc3ccccc3)c2)C1]

Sodium triacetoxyborohydride (5.12 g; 24.15 mmol; 1 eq.) was added to a suspension of pyrrolidine-3-carboxylic acid methyl ester hydrochloride (4 g; 24.15 mmol; 1 eq.) and 3-phenoxy-benzaldehyde (4.79 g; 24.15 mmol; 1 eq.) in DCE (15 mL) and the resulting mixture was stirred at 65° C. for 2 hours then partitioned between DCM and 0.1M NaOH. The two phases were separated and the aqueous layer extracted with DCM. The combined organics were dried over sodium sulfate and concentrated in vacuo. Purification by column chromatography (20% EA in heptane) afforded the title compound (2.6 g, 35%) as brown oil. HPLC (max plot) 88% Rt 2.98 min. LC/MS: (MS+) 312.3 (M+H⁺).

### Intermediate C1: (R)-pyrrolidine-3-carboxylic acid (pyridin-2-ylmethyl)-amide hydrochloride

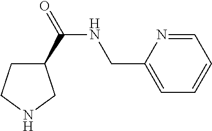 [SMILES: O=C(NCc1ccccn1)[C@@H]1CCNC1]

A mixture of Intermediate Fl (600 mg; 1.96 mmol; 1 eq.) and 4M HCl in 1,4-dioxane (10 mL; 40 mmol; 20.4 eq.) was stirred at room temperature for 16 hours then concentrated in vacuo. The residue was triturated in Et₂O and the precipitate filtered off to afford the title compound (350 mg, 74%) as white solid. HPLC (max plot) 69% Rt 1.41 min. LC/MS (MS+) 271.3 (M+H⁺).

### Intermediate D1: (±) 3-methyl-pyrrolidine-3-carboxylic acid methyl ester hydrochloride

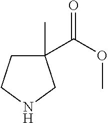

Thionyl chloride (1.13 mL; 15.5 mmol; 1 eq.) was added at 0° C. to MeOH (50 mL) and the reaction mixture was stirred at room temperature for 15 minutes whereupon 3-methyl-pyrrolidine-3-carboxylic acid (2 g; 15.5 mmol; 1 eq.) was added. The resulting mixture was stirred at room temperature for 16 hours then concentrated in vacuo to afford the title compound (2.65 g, 95%) as yellow oil. ¹H NMR (300 MHz, DMSO-d₆) δ=9.79-9.27 (br s, 2H), 3.67 (s, 3H), 3.51 (d, J=11.9 Hz, 1H), 3.36-3.11 (m, 2H), 3.03 (d, J=11.9 Hz, 1H), 2.38-2.19 (m, 1H), 1.93-1.76 (m, 1H), 1.33 (s, 3H).

### Intermediate E1: 2-phenyl-thiazole-4-carbaldehyde

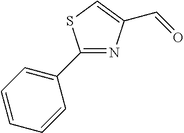 [SMILES: O=Cc1csc(-c2ccccc2)n1]

Manganese^{(Iv)} oxide (1.59 g; 18.3 mmol; 7 eq.) was added to a solution of (2-phenyl-thiazol-4-yl)-methanol (500 mg; 2.61 mmol; 1 eq.) and in DCM (20 mL) and the reaction mixture was stirred at room temperature for 18 hours. The suspension was filtered through a plug of Celite® and the solution concentrated in vacuo to afford the title compound (295 mg, 60%) as yellow oil. LC/MS (MS+) 190.0 (M+H⁺).

### Intermediate F1: (R)-3-[(pyridin-2-ylmethyl)-carbamoyl]-pyrrolidine-1-carboxylic acid tert-butyl ester

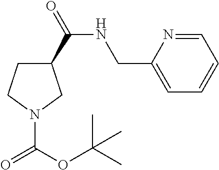 [SMILES: CC(C)(C)OC(=O)N1CC[C@@H](C(=O)NCc2ccccn2)C1]

EDC (490 mg; 2.56 mmol; 1.1 eq.) was added to a solution of (R)-pyrrolidine-1,3-dicarboxylic acid 1-tert-butyl ester (500 mg; 2.32 mmol; 1 eq.) in DCM (20 mL) and the resulting mixture was stirred at room temperature for 10 minutes whereupon pyridin-3-yl-methylamine (0.31 μL; 3.02 mmol; 1.3 eq.) was added. The reaction mixture was stirred at room temperature for 16 hours then washed with sat. aq. Na₂CO₃ and 10% aq. citric acid, dried over magnesium sulfate and concentrated in vacuo to afford the title compound (600 mg, 85%) as colourless oil. HPLC (max plot) 99% Rt 1.97 min. LC/MS: (MS+) 306.3 (M+H⁺)

Examples 1, 2, 4, 5, 6, 7 and 8 were obtained according to or in analogy to the methods described herein. In one embodiment of the present invention compounds other than that of examples 1, 2, 4, 5, 6, 7 and 8 are preferred.

### Example 9

### (±) 3-methyl-1-(3-phenoxy-benzyl)-pyrrolidine-3-carboxylic acid 2-methyl-benzylamide

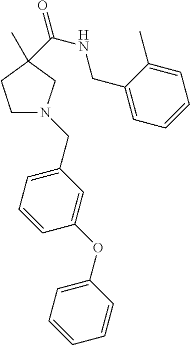 [SMILES: Cc1ccccc1CNC(=O)C1(C)CCN(Cc2cccc(Oc3ccccc3)c2)C1]

A 50% solution of T3P in EA (307 mg; 0.48 mmol; 1.5 eq.) was added to a solution of Intermediate A1 (100 mg; 0.32 mmol; 1 eq.) 2-methyl-benzylamine (40 μL; 0.35 mmol; 1.1 eq.) and TEA (140 μL; 0.96 mmol; 3 eq.) in EA (10 mL) and the reaction mixture was stirred at room temperature for 1 hour. Purification by column chromatography (EA to 10% MeOH in EA) afforded the title compound (40 mg, 30.1%) as colourless oil. ¹H NMR (300 MHz, CDCl₃) δ=8.04 (s, 1H), 7.32 (dd, J=8.6, 7.3 Hz, 2H), 7.24-7.04 (m, 6H), 7.04-6.90 (m, 2H), 6.90-6.75 (m, 2H), 6.69 (d, J=7.6 Hz, 1H), 4.39 (dd, J=14.8, 5.4 Hz, 1H), 4.30 (dd, J=14.8, 5.4 Hz, 2H), 3.63-3.45 (m, 2H), 3.11-2.90 (m, 2H), 2.35-2.26 (m, 4H), 2.20-2.05 (m, 2H), 1.73 (ddd, J=13.1, 9.5, 2.9 Hz, 1H), 1.28 (s, 2H). HPLC (max plot) 90.6% Rt 3.85 min. LC/MS: (MS+) 415.5 (M+H⁺).

### Example 10

### (S)-3-methyl-1-(3-phenoxy-benzyl)-pyrrolidine-3-carboxylic acid ethylamide

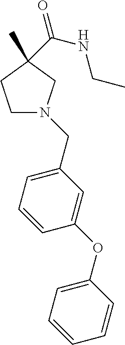 [SMILES: CCNC(=O)[C@@]1(C)CCN(Cc2cccc(Oc3ccccc3)c2)C1]

A 50% solution of T3P in EA (920 mg; 1.45 mmol; 1.5 eq.) was added to a solution of 3-methyl-1-(3-phenoxy-benzyl)-pyrrolidine-3-carboxylic acid (300 mg; 0.96 mmol; 1 eq.), ethylamine (60 mL; 1.06 mmol; 1.1 eq.) and TEA (420 μL; 2.89 mmol; 3 eq.) in EA (10 mL) and the resulting mixture was stirred at room temperature for 1 hour. Purification by column chromatography (EA to 10% MeOH in EA) gave the racemate compound (150 mg, 43%). A chiral separation on 110 mg of this racemate (SFC separation, 0.5% DEA in MeOH, OJ-H 80 mL/min, first eluting enantiomer) afforded the title compound (50 mg, 15%) as colourless oil. ¹H NMR (300 MHz, CDCl₃) δ=7.63 (br s, 1H), 7.41-7.23 (m, 3H), 7.13 (td, J=7.2, 1.2 Hz, 1H), 7.08-6.96 (m, 4H), 6.96-6.87 (m, 1H), 3.76-3.53 (m, 1H), 3.49 (q, J=6.7 Hz, 1H), 3.26-3.14 (m, 2H), 3.12-2.97 (m, 2H), 2.37 (d, J=8.9 Hz, 1H), 2.21-1.96 (m, 2H), 1.72 (s, 1H), 1.26 (s, 2H), 1.22 (t, J=6.5 Hz, 2H), 1.07 (t, J=6.5 Hz, 3H). HPLC (max plot) 96%; Rt 3.00 min. LC/MS (MS+) 339.3 (M+H⁺).

### Example 11

### (R)-3-methyl-1-(3-phenoxy-benzyl)-pyrrolidine-3-carboxylic acid ethylamide

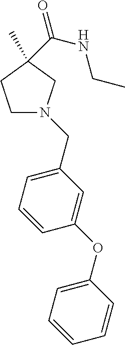 [SMILES: CCNC(=O)[C@]1(C)CCN(Cc2cccc(Oc3ccccc3)c2)C1]

The title compound (50 mg, 15%) was isolated as colourless oil the second eluting enantiomer during the chiral separation performed for the preparation of Example 10. ¹H NMR (300 MHz, CDCl₃) δ=7.63 (br s, 1H), 7.41-7.23 (m, 3H), 7.13 (td, J=7.2, 1.2 Hz, 1H), 7.08-6.96 (m, 4H), 6.96-6.87 (m, 1H), 3.76-3.53 (m, 1H), 3.49 (q, J=6.7 Hz, 1H), 3.26-3.14 (m, 2H), 3.12-2.97 (m, 2H), 2.37 (d, J=8.9 Hz, 1H), 2.21-1.96 (m, 2H), 1.72 (s, 1H), 1.26 (s, 2H), 1.22 (t, J=6.5 Hz, 2H), 1.07 (t, J=6.5 Hz, 3H). HPLC (max plot) 99%, Rt 3.01 min. LC/MS (MS+) 339.3 (M+H⁺).

### Example 12

### (±) 3-methyl-1-(3-phenoxy-benzyl)-pyrrolidine-3-carboxylic acid (pyridin-2-ylmethyl)-amide

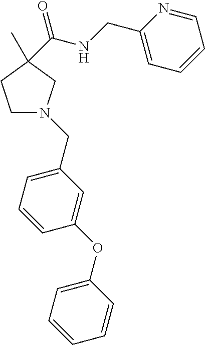 [SMILES: CC1(C(=O)NCc2ccccn2)CCN(Cc2cccc(Oc3ccccc3)c2)C1]

HATU (458 mg; 1.2 mmol; 1.5 eq.) was added to a solution of Intermediate A1 (250 mg; 0.80 mmol; 1 eq.) and TEA (390 μL; 2.41 mmol; 3 eq.) in DMF (5 mL) and the resulting mixture was stirred at room temperature for 30 minutes whereupon pyridin-2-yl-methylamine (80 μL; 0.80 mmol; 1 eq.) was added. The reaction mixture was stirred at room temperature for 2 hours then concentrated in vacuo. Purification by mass directed preparative HPLC afforded the title compound (200 mg; 62%) as brown oil. ¹H NMR (DMSO-d₆, 300 MHz) δ=8.54-8.43 (m, 1H), 8.37 (s, 1H), 7.72 (td, J=7.8, 1.8 Hz, 1H), 7.55-6.73 (m, 10H), 4.35 (d, J=5.7 Hz, 3H), 3.76-3.44 (m, 2H), 2.99-2.54 (m, 3H), 2.39-2.19 (m, 2H), 1.73-1.49 (m, 1H), 1.28 (s, 3H). HPLC (max plot) 98%, Rt (min) 2.32. LC/MS (MS+) 402.3 (M+H⁺).

### Example 13

### (±) [3-methyl-1-(3-phenoxy-benzyl)-pyrrolidin-3-yl]-(8-trifluoromethyl-3,4-dihydro-1H-isoquinolin-2-yl)-methanone

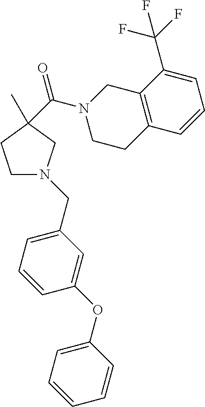 [SMILES: CC1(C(=O)N2CCc3cccc(C(F)(F)F)c3C2)CCN(Cc2cccc(Oc3ccccc3)c2)C1]

HATU (733 mg; 1.93 mmol; 1.5 eq.) was added to a solution of Intermediate A1 (400 mg; 1.28 mmol; 1 eq.) and TEA (660 μL; 3.85 mmol; 3 eq.) in DMF (10 mL) and the resulting mixture was stirred at room temperature for 20 minutes whereupon 8-trifluoromethyl-1,2,3,4-tetrahydro-isoquinoline hydrochloride (305 mg; 1.28 mmol; 1.00 eq.) was added. The reaction mixture was stirred at room temperature for 1.5 hours then diluted with water. The solution was extracted with DCM (3×). The combined organic phase was dried over sodium sulfate and concentrated in vacuo. Purification by mass directed preparative HPLC afforded the title compound (230 mg; 36%) as brown oil. ¹H NMR (DMSO-d₆, 300 MHz) δ=7.59 (d, J=7.2 Hz, 1H), 7.54-7.25 (m, 5H), 7.20-6.96 (m, 4H), 6.97-6.85 (m, 2H), 4.79 (q, J=17.4 Hz, 2H), 3.93-3.59 (m, 2H), 3.53 (br s, 2H), 3.07-2.80 (m, 3H), 2.73-2.55 (m, 1H), 2.46-2.18 (m, 3H), 1.75 (br s, 1H), 1.26 (br s, 3H). HPLC (max plot) 98%, Rt 4.21 min. LC/MS (MS-F) 495.3 (M+H⁺).

### Example 14

### (±) 3-methyl-1-(3-phenoxy-benzyl)-pyrrolidine-3-carboxylic acid methyl-pyridin-2-ylmethyl-amide

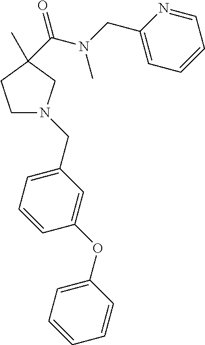 [SMILES: CN(Cc1ccccn1)C(=O)C1(C)CCN(Cc2cccc(Oc3ccccc3)c2)C1]

Intermediate A1 (250 mg; 0.8 mmol; 1 eq.) and HATU (458 mg; 1.2 mmol; 1.5 eq.) were stirred during 15 min in DMF (5 mL), then methyl-pyridin-2-ylmethyl-amine (98 mg, 0.80 mmol, 1.0 equiv.) was added to the mixture. The reaction was stirred at rt during 1.5 h and was then diluted with water. The solution was extracted with DCM (3×). The combined organic phase was dried over sodium sulfate and concentrated in vacuo. Purification by mass directed preparative HPLC afforded the title compound (150 mg, 45%) of the title compound as brown solid. ¹H NMR (300 MHz, DMSO-d₆) δ=8.51 (d, J=4.2 Hz, 1H), 7.77 (t, J=6.9 Hz, 1H), 7.52-6.74 (m, 11H), 4.58 (br s, 2H), 3.91-3.18 (m, 2H), 3.17-2.57 (m, 4H), 2.46-1.55 (m, 3H), 1.38 (br s, 3H). LC/MS (MS+) 416.5 (M+Hⁱ).

### Example 15

### (±) [3-methyl-1-(3-phenoxy-benzyl)-pyrrolidin-3-yl]-morpholin-4-yl-methanone

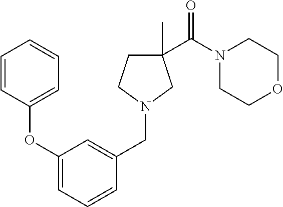 [SMILES: CC1(C(=O)N2CCOCC2)CCN(Cc2cccc(Oc3ccccc3)c2)C1]

A 50% solution of T3P in EA (240 μL; 0.77 mmol; 3 eq.) was added to a cold (0° C.) solution of Intermediate A1 (80 mg, 0.25 mmol; 1 eq.), morpholine (34 μL, 0.38 mmol; 1.5 eq.) and TEA (100 μL; 0.77 mmol; 3 eq.) in DCM (3 mL) and the reaction mixture was stirred at room temperature for 5 hours. The solution was washed with water and the organic layer filtered through a SPE-NH₂ column to afford the title compound (7.2 mg, 8.4%) as colourless oil. ¹H NMR (400 MHz, DMSO-d₆) δ=7.40-7.36 (m, 2H), 7.33-7.30 (m, 1H), 7.15-7.11 (m, 1H), 7.05 (d, J=7.6 Hz, 1H), 7.00 (d, J=7.7 Hz, 2H), 6.92 (s, 1H), 6.87 (dd, J=2.1, 8.0 Hz, 1H), 3.51-3.47 (m, 6H), 3.45-3.42 (m, 3H), 2.89-2.86 (m, 1H), 2.62-2.58 (m, 1H), 2.40-2.35 (m, 1H), 2.31 (d, J=9.4 Hz, 1H), 2.22-2.16 (m, 1H), 2.02-1.96 (m, 1H), 1.71-1.65 (m, 1H), 1.23 (s, 3H). HPLC (max plot) 93% Rt 3.35 min. LC/MS: (MS+) 381.2 (M+Fr).

### Example 16

### (±) 3-methyl-1-(3-phenoxy-benzyl)-pyrrolidine-3-carboxylic acid (pyridin-3-ylmethyl)-amide

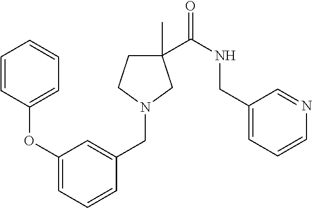 [SMILES: CC1(C(=O)NCc2cccnc2)CCN(Cc2cccc(Oc3ccccc3)c2)C1]

A 50% solution of T3P in EA (240 μL; 0.77 mmol; 3 eq.) was added to a cold (0° C.) solution of Intermediate A1 (80 mg, 0.25 mmol; 1 eq.), 3-(aminomethyl)pyridine (41 mg, 0.38 mmol; 1.5 eq.) and TEA (100 μL; 0.77 mmol; 3 eq.) in DCM (3 mL) and the reaction mixture was stirred at room temperature for 5 hours. The solution was washed with water and the organic layer filtered through a SPE-NH₂ column to afford the title compound (15.9 mg, 18%) as colourless oil. ¹H NMR (400 MHz, DMSO-d₆) δ=8.42-8.41 (m, 2H), 8.24-8.21 (m, 1H), 7.58-7.56 (m, 1H), 7.39-7.35 (m, 2H), 7.32-7.27 (m, 2H), 7.14-7.10 (m, 1H), 7.04 (d, J=4.8 Hz, 1H), 6.98 (dd, J=1.0, 1.9 Hz, 2H), 6.94-6.93 (m, 1H), 6.87-6.85 (m, 1H), 4.26-4.24 (m, 2H), 3.58-3.50 (m, 2H), 2.78 (d, J=9.2 Hz, 1H), 2.56-2.53 (m, 2H), 2.32-2.21 (m, 2H), 1.56-1.51 (m, 1H), 1.28-1.21 (m, 3H). HPLC (max plot) 95% Rt 2.84 min. LC/MS: (MS+) 402.2 (M+H⁺).

### Example 17

### (±) 3-methyl-1-(3-phenoxy-benzyl)-pyrrolidine-3-carboxylic acid (pyridin-4-ylmethyl)-amide

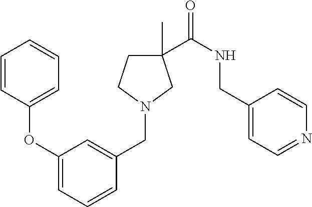 [SMILES: CC1(C(=O)NCc2ccncc2)CCN(Cc2cccc(Oc3ccccc3)c2)C1]

A 50% solution of T3P in EA (240 μL; 0.77 mmol; 3 eq.) was added to a cold (0° C.) solution of Intermediate A1 (80 mg, 0.25 mmol; 1 eq.), 4-(aminomethyl)pyridine (41 mg, 0.38 mmol; 1.5 eq.) and TEA (100 μL; 0.77 mmol; 3 eq.) in DCM (3 mL) and the reaction mixture was stirred at room temperature for 5 hours. The solution was washed with water and the organic layer filtered through a SPE-NH₂ column to afford the title compound (15.9 mg, 18%) as colourless oil. ¹H NMR (400 MHz, DMSO-d₆) δ=8.45 (dd, J=1.5, 4.5 Hz, 2H), 8.27-8.24 (m, 1H), 7.39-7.34 (m, 2H), 7.32-7.28 (m, 1H), 7.17-7.14 (m, 2H), 7.12-7.10 (m, 1H), 7.05 (d, J=7.6 Hz, 1H), 7.00-6.96 (m, 3H), 6.89-6.83 (m, 1H), 4.26-4.24 (m, 2H), 3.59-3.52 (m, 2H), 2.83 (d, J=9.2 Hz, 1H), 2.59-2.51 (m, 2H), 2.31-2.25 (m, 2H), 1.58-1.51 (m, 1H), 1.24-1.23 (m, 3H). HPLC (max plot) 97% Rt 2.82 min. LC/MS: (MS+) 402.2 (M+H⁺).

### Example 18

### (±) 1-(3-phenoxy-benzyl)-pyrrolidine-3-carboxylic acid ethylamide

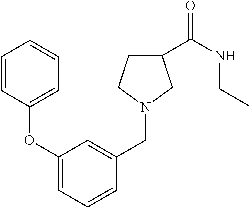 [SMILES: CCNC(=O)C1CCN(Cc2cccc(Oc3ccccc3)c2)C1]

A 50% solution of T3P in EA (340 μL; 1 mmol; 3 eq.) was added to a cold (0° C.) solution of Intermediate A3 (100 mg, 0.33 mmol; 1 eq.), 2M ethylamine in THF (250 μL, 0.5 mmol; 1.5 eq.) and TEA (140 μL; 1 mmol; 3 eq.) in DCM (3 mL) and the reaction mixture was stirred at room temperature for 5 hours. The solution was washed with water and the organic layer filtered through a SPE-NH₂ column to afford the title compound (27 mg, 30%) as colourless oil. ¹H NMR (400 MHz, DMSO-d₆) δ=7.73 (t, J=5.2 Hz, 1H), 7.38-7.41 (m, 2H), 7.36 (t, J=4.2 Hz, 1H), 7.13 (t, J=7.4 Hz, 1H), 7.06 (d, J=7.6 Hz, 1H), 6.99 (dd, J=0.8, 8.6 Hz, 2H), 6.93 (s, 1H), 6.86 (dd, J=1.7, 7.7 Hz, 1H), 3.53 (s, 2H), 2.99-3.05 (m, 2H), 2.71-2.78 (m, 2H), 2.58-2.61 (m, 1H), 2.32-2.36 (m, 2H), 1.84-1.88 (m, 2H), 0.97 (t, J=7.2 Hz, 3H). HPLC (max plot) 97% Rt 3.29 min. LC/MS: (MS+) 325.3 (M+H⁺).

### Example 19

### (±) 1-(3-phenoxy-benzyl)-pyrrolidine-3-carboxylic acid (2-methoxy-ethyl)-amide

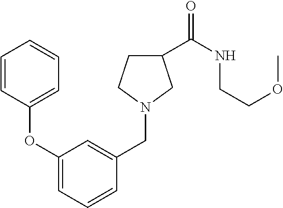 [SMILES: COCCNC(=O)C1CCN(Cc2cccc(Oc3ccccc3)c2)C1]

A 50% solution of T3P in EA (340 μL; 1 mmol; 3 eq.) was added to a cold (0° C.) solution of Intermediate A3 (100 mg, 0.33 mmol; 1 eq.), 2-methoxyethylamine (38 mg, 0.5 mmol; 1.5 eq.) and TEA (140 μL; 1 mmol; 3 eq.) in DCM (3 mL) and the reaction mixture was stirred at room temperature for 5 hours. The solution was washed with water and the organic layer filtered through a SPE-NH₂ column to afford the title compound (33 mg, 39%) as colourless oil. ¹H NMR (400 MHz, DMSO-d₆) δ=7.85-7.82 (m, 1H), 7.40-7.36 (m, 2H), 7.33-7.29 (m, 1H), 7.14-7.11 (m, 1H), 7.06 (d, J=7.6 Hz, 1H), 6.99 (dd, J=0.8, 8.6 Hz, 2H), 6.94 (s, 1H), 6.86 (dd, J=1.6, 8.1 Hz, 1H), 3.53 (s, 2H), 3.29 (d, J=5.6 Hz, 2H), 3.21 (s, 3H), 3.19-3.14 (m, 2H), 2.82-2.77 (m, 1H), 2.74-2.70 (m, 1H), 2.62-2.56 (m, 1H), 2.39-2.33 (m, 2H), 1.88-1.82 (m, 2H). HPLC (max plot) 97% Rt 3.21 min. LC/MS: (MS+) 355.3 (M+H⁺).

### Example 20

### (±) [3-methyl-1-(3-phenoxy-benzyl)-pyrrolidin-3-yl]-(4-methyl-piperazin-1-yl)-methanone

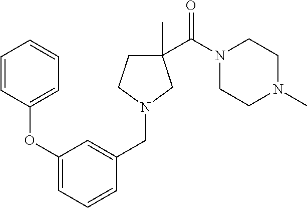 [SMILES: CN1CCN(C(=O)C2(C)CCN(Cc3cccc(Oc4ccccc4)c3)C2)CC1]

A 50% solution of T3P in EA (240 μL; 0.77 mmol; 3 eq.) was added to a cold (0° C.) solution of Intermediate A1 (80 mg, 0.25 mmol; 1 eq.), 1-methylpiperazine (38 mg, 0.38 mmol; 1.5 eq.) and TEA (100 μL; 0.77 mmol; 3 eq.) in DCM (3 mL) and the reaction mixture was stirred at room temperature for 5 hours. The solution was washed with water and the organic layer filtered through a SPE-NH₂ column to afford the title compound (3.4 mg, 4%) as colourless oil. ¹H NMR (400 MHz, DMSO-d₆) δ=7.40-7.39 (m, 2H), 7.33-7.29 (m, 1H), 7.15-7.11 (m, 1H), 7.05-7.04 (m, 1H), 7.00 (dd, J=0.8, 8.6 Hz, 2H), 6.92-6.88 (m, 1H), 6.88-6.86 (m, 1H), 3.51 (s, 2H), 3.43-3.42 (m, 4H), 2.86 (d, J=9.0 Hz, 1H), 2.50-2.48 (m, 1H), 2.39-2.32 (m, 1H), 2.30-2.27 (m, 1H), 2.24-2.16 (m, 4H), 2.15 (s, 3H), 2.02-1.96 (m, 1H), 1.71-1.23 (m, 1H), 1.23 (s, 3H). HPLC (max plot) 95% Rt 2.83 min. LC/MS: (MS+) 394.2 (M+H⁺).

### Example 21

### (±) 3-methyl-1-(2-phenyl-thiazol-4-ylmethyl)-pyrrolidine-3-carboxylic acid (pyridin-2-ylmethyl)-amide

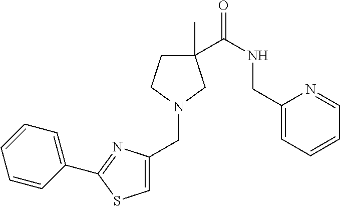 [SMILES: CC1(C(=O)NCc2ccccn2)CCN(Cc2csc(-c3ccccc3)n2)C1]

HATU (151 mg; 0.4 mmol; 1.2 eq.) was added to a solution of Intermediate A2 (100 mg; 0.33 mmol; 1 eq.) and TEA (90 μL; 0.66 mmol; 2 eq.) in DMF (4 mL) and the resulting mixture was stirred at room temperature for 15 minutes whereupon pyridin-2-yl-methylamine (36 mg; 0.33 mmol; 1 eq.) was added. The reaction mixture was stirred at room temperature for 1 hour then diluted with 0.1M NaOH. The solution was extracted with EA (3×). The combined organic phase was dried over magnesium sulfate and concentrated in vacuo. Purification by mass directed preparative HPLC afforded the title compound (66 mg; 51%) as yellow oil. ¹H NMR (300 MHz, DMSO-d₆) δ=8.46 (ddd, J=4.8, 1.8, 0.9 Hz, 1H), 8.40 (t, J=5.9 Hz, 1H), 7.97-7.83 (m, 2H), 7.68 (td, J=7.7, 1.8 Hz, 1H), 7.53 (s, 1H), 7.51-7.41 (m, 3H), 7.28-7.12 (m, 2H), 4.36 (dd, J=5.5, 1.6 Hz, 2H), 3.88-3.78 (m, 1H), 3.78-3.68 (m, 1H), 3.07 (d, J=9.3 Hz, 1H), 2.80 (td, J=8.6, 5.0 Hz, 1H), 2.64 (td, J=8.5, 6.3 Hz, 1H), 2.39 (d, J=9.2 Hz, 1H), 2.36-2.22 (m, 1H), 1.61 (ddd, J=13.0, 8.3, 5.0 Hz, 1H), 1.27 (s, 3H). HPLC (max plot) 97% Rt 2.00 min. LC/MS: (MS+) 393.1 (M+H⁺).

### Example 22

### (±) 1-(3-isobutoxy-benzyl)-3-methyl-pyrrolidine-3-carboxylic acid methyl ester

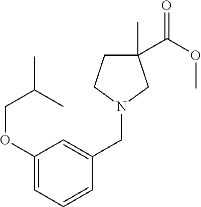 [SMILES: COC(=O)C1(C)CCN(Cc2cccc(OCC(C)C)c2)C1]

Sodium triacetoxyborohydride (307 mg; 1.45 mmol; 1.3 eq.) was added to a suspension of Intermediate B1 (200 mg; 1.11 mmol; 1 eq.) and 3-isobutoxy-benzaldehyde (218 mg; 1.22 mmol; 1.1 eq.) in DCE (2 mL) and the resulting mixture was stirred at 65° C. for 30 minutes then partitioned between DCM and 0.1M NaOH. The two phases were separated and the aqueous layer extracted with DCM. The combined organics were dried over sodium sulfate and concentrated in vacuo. Purification by column chromatography (10% to 20% EA in heptane) afforded the title compound (175 mg, 51%) as colourless oil. ¹H NMR (300 MHz, DMSO-d₆) δ=7.24-7.14 (m, 1H), 6.88-6.70 (m, 3H), 3.71 (d, J=6.5 Hz, 2H), 3.61 (s, 3H), 3.51 (d, J=3.0 Hz, 2H), 2.86 (d, J=9.2 Hz, 1H), 2.63-2.51 (m, 2H), 2.33-2.22 (m, 2H), 2.08-1.90 (m, 1H), 1.68-1.50 (m, 1H), 1.26 (s, 3H), 0.97 (d, J=6.7 Hz, 6H). HPLC (max plot) 100% Rt 3.26 min. LC/MS: (MS+) 306.4 (M+H⁺).

### Example 23

### (±) 1-(3-isobutoxy-benzyl)-3-methyl-pyrrolidine-3-carboxylic acid (pyridin-2-ylmethyl)-amide

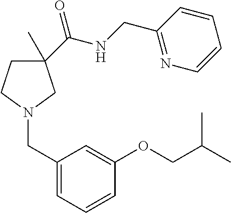 [SMILES: CC(C)COc1cccc(CN2CCC(C)(C(=O)NCc3ccccn3)C2)c1]

A 2M solution of trimethylaluminum in heptane (0.59 mL; 1.18 mmol; 4 eq.) was added to a cold (0° C.) solution of pyridin-2-yl-methylamine (64 mg; 0.59 mmol; 2 eq.) in DCE (2 mL) and the resulting mixture was stirred at 0° C. for 15 whereupon a solution of Example 22 (90 mg; 0.29 mmol; 1 eq.) in DCE (1 mL) was added. The reaction mixture was stirred at 65° C. for 48 hours then diluted with DCM and washed with an aqueous solution of Rochelle's salt. The organic layer was dried over sodium sulfate and concentrated in vacuo. Purification by column chromatography (EA to 3% MeOH in EA) afforded the title compound (70 mg, 62%) as yellow oil. ¹H NMR (300 MHz, DMSO-d₆) δ=8.55-8.46 (m, 1H), 8.43-8.34 (m, 1H), 7.78-7.68 (td, J=7.7, 1.9 Hz, 1H), 7.29-7.13 (m, 3H), 6.94-6.84 (m, 2H), 6.81-6.74 (dd, J=8.0, 2.4 Hz, 1H), 4.40-4.32 (m, 2H), 3.67 (d, J=6.5 Hz, 2H), 3.58-3.52 (m, 2H), 2.92 (d, J=9.3 Hz, 1H), 2.73-2.61 (s, 1H), 2.35-2.18 (m, 1H), 2.05-1.88 (m, 1H), 1.64-1.52 (m, 1H), 1.26 (s, 3H), 0.97-0.88 (d, J=6.7 Hz, 6H). HPLC (max plot) 99% Rt 2.51 min. LC/MS: (MS+) 382.4 (M+H⁺).

### Example 24

### (R)-1(3-phenoxy-benzyl)-pyrrolidine-3-carboxylic acid (pyridin-3-ylmethyl)-amide

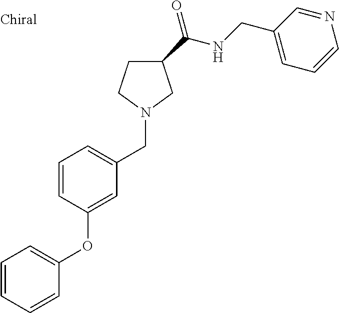 [SMILES: O=C(NCc1cccnc1)[C@@H]1CCN(Cc2cccc(Oc3ccccc3)c2)C1]

Sodium triacetoxyborohydride (189 mg; 0.89 mmol; 1.2 eq.) was added to a suspension of Intermediate C1 (180 mg; 0.74 mmol; 1 eq.) and 3-phenoxy-benzaldehyde (177 mg; 0.89 mmol; 1 eq.) in DCE (3 mL) and THF (3 mL) and the resulting mixture was stirred at 75° C. for 24 hours then partitioned between DCM and 0.1M NaOH. The two phases were separated and the aqueous layer extracted with DCM. The combined organics were dried over sodium sulfate and concentrated in vacuo. Purification by column chromatography (30% EA in heptane) afforded the title compound (20 mg, 5%) as colourless oil. HPLC (max plot) 97% Rt 2.40 min. LC/MS: (MS+) 388.3 (M+H⁺).

### Example 25

### (±) 1-(3-isobutoxy-benzyl)-3-methyl-pyrrolidine-3-carboxylic acid methyl-pyridin-2-ylmethyl-amide

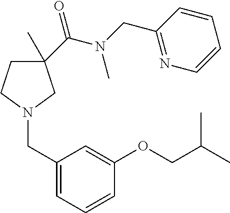 [SMILES: CC(C)COc1cccc(CN2CCC(C)(C(=O)N(C)Cc3ccccn3)C2)c1]

A 2M solution of trimethylaluminum in heptane (0.74 mL; 1.5 mmol; 5 eq.) was added to a cold (0° C.) solution of methyl-pyridin-2-ylmethyl-amine (108 mg; 0.88 mmol; 3 eq.) in DCE (2 mL) and the resulting mixture was stirred at 0° C. for 10 minutes whereupon Intermediate B1 (90 mg; 0.29 mmol; 1 eq.) was added. The reaction mixture was stirred at 65° C. for 72 hours then diluted with DCM and washed with an aqueous solution of Rochelle's salt. The organic layer was dried over sodium sulfate and concentrated in vacuo. Purification by column chromatography (EA to 5% MeOH in EA) afforded the title compound (10 mg, 9%) as yellow oil. HPLC (max plot) 76% Rt 2.58 min. LC/MS: (MS+) 396.4 (M+H⁺).

### Example 26

### (±) 3-methyl-1-(3-phenoxy-benzyl)-pyrrolidine-3-carboxylic acid (2-pyridin-2-v1-ethyl)-amide

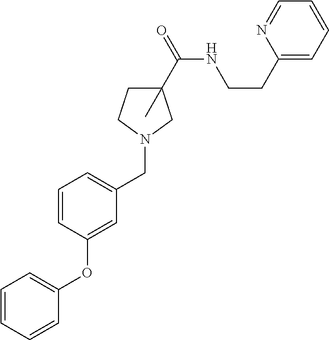

A 2M solution of trimethylaluminum in heptane (0.74 mL; 1.5 mmol; 5 eq.) was added to a cold (0° C.) solution of methyl-pyridin-2-ylmethyl-amine (108 mg; 0.88 mmol; 3 eq.) in DCE (2 mL) and the resulting mixture was stirred at 0° C. for 10 minutes whereupon Intermediate B1 (90 mg; 0.29 mmol; 1 eq.) was added. The reaction mixture was stirred at 65° C. for 72 hours then diluted with DCM and washed with an aqueous solution of Rochelle's salt. The organic layer was dried over sodium sulfate and concentrated in vacuo. Purification by column chromatography (EA to 5% MeOH in EA) afforded the title compound 7 mg (5%) as colorless oil. HPLC (max plot) 100% Rt 2.45 min. LC/MS: (MS+) 416.3 (M+H⁺).

### Example 27

### (±) (2-methyl-morpholin-4-yl)-[1-(3-phenoxy-benzyl)-pyrrolidin-3-yl]-methanone

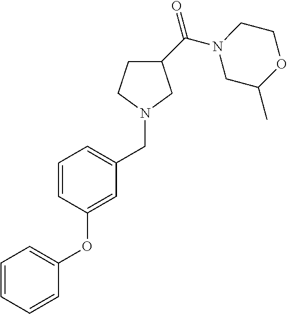 [SMILES: CC1CN(C(=O)C2CCN(Cc3cccc(Oc4ccccc4)c3)C2)CCO1]

Bis(trimethylaluminum)-1,4-diazabicyclo(2.2.2)octane complex (206 mg; 0.8 mmol; 2.5 eq.) was added to a solution of 2-methyl-morpholine (81 mg; 0.8 mmol; 2.5 eq.) in THF (5 mL) and the resulting mixture was stirred at room temperature for 5 minutes whereupon a solution of Intermediate B3 (100 mg; 0.32 mmol; 1 eq.) in THF (5 mL) was added. The reaction mixture was stirred at 55° C. for 48 hours then partitioned between DCM and water. The two phases were separated and the aqueous layer extracted with DCM. The combined organics were dried over sodium sulfate and concentrated in vacuo. Purification by column chromatography (EA/EtOH/NH₄OH, 97/3/1) afforded the title compound (91 mg, 74%) as colourless oil and as a mixture of mixture of diastereoisomers. ¹H NMR (300 MHz, DMSO-d₆) δ=7.34-7.23 (m, 3H), 7.11-7.06 (m, 2H), 7.03-6.96 (m, 3H), 6.88 (d, J=8 Hz, 1H), 4.44 (t, J=7.5 Hz, 1H), 3.92-3.84 (m, 1H), 3.70-3.61 (m, 3H), 3.52-3.40 (m, 2H), 3.22-3.10 (m, 1H), 2.98-2.71 (m, 3H), 2.54-2.38 (m, 3H), 1.98-1.94 (m, 2H), 1.18 (d, J=9.0 Hz, 3H). HPLC (max plot) 100% Rt 3.01 min. LC/MS: (MS+) 381.4 (M+H⁺).

### Example 28

### (±) 1-(3-phenoxy-benzyl)-pyrrolidine-3-carboxylic acid (2-pyridin-2-yl-ethyl)-amide

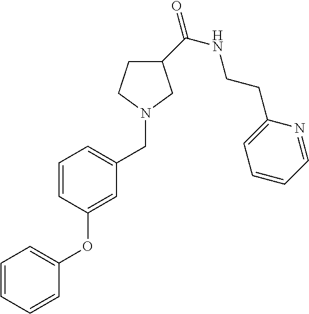 [SMILES: O=C(NCCc1ccccn1)C1CCN(Cc2cccc(Oc3ccccc3)c2)C1]

Bis(trimethylaluminum)-1,4-diazabicyclo(2.2.2)octane complex (206 mg; 0.8 mmol; 2.5 eq.) was added to a solution of 2-pyridin-2-yl-ethylamine (81 mg; 0.8 mmol; 2.5 eq.) in THF (5 mL) and the resulting mixture was stirred at room temperature for 5 minutes whereupon a solution of Intermediate B3 (100 mg; 0.32 mmol; 1 eq.) in THF (5 mL) was added. The reaction mixture was stirred at 65° C. for 48 hours then partitioned between DCM and an aqueous solution of Rochelle's salt. The two phases were separated and the aqueous layer extracted with DCM. The combined organics were dried over sodium sulfate and concentrated in vacuo. Purification by column chromatography (5% MeOH in EA) afforded the title compound (91 mg, 74%) as colourless oil. ¹H NMR (300 MHz, DMSO-d₆) δ=8.47-8.45 (m, 1H), 7.57-7.52 (m, 1H), 7.37-7.21 (m, 4H), 7.11-7.04 (m, 3H), 6.99-6.85 (m, 4H), 3.62-3.48 (m, 4H), 2.92 (t, J=8 Hz, 2H), 2.82-2.66 (m, 3H), 2.50-2.32 (m, 2H), 2.14-1.81 (m, 2H). HPLC (max plot) 100% Rt 2.42 min. LC/MS: (MS+) 402.4 (M+H⁺).

### Example 29

### (±) (3-methoxy-piperidin-1-yl)-[1-(3-phenoxy-benzyl)-pyrrolidin-3-yl]-methanone

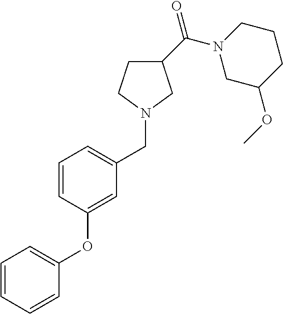 [SMILES: COC1CCCN(C(=O)C2CCN(Cc3cccc(Oc4ccccc4)c3)C2)C1]

A 50% solution of T3P in EA (112 mg; 0.35 mmol; 1.5 eq.) was added to a solution of Intermediate A3 (70 mg; 0.24 mmol; 1 eq.), 3-methoxy-piperidine hydrochloride (54 mg; 0.35 mmol; 1.5 eq.) and TEA (95 mg; 0.94 mmol; 4 eq.) in THF (10 mL) and the resulting mixture was stirred at room temperature for 1 hour. The solution was partitioned between water and DCM and the two phases separated. The aqueous layer was extracted with DCM. The combined organics were dried over sodium sulfate and concentrated in vacuo. Purification by column chromatography (EA/MeOH/NR₄OH, 95/5/1) afforded the title compound (26 mg, 28%) as a colourless oil and as a mixture of diastereosiomers. ¹H NMR (300 MHz, DMSO-d₆) δ=7.31-7.25 (m, 3H), 7.12-6.99 (m, 5H), 6.89 (d, J=8 Hz, 1H), 3.94-3.93 (m, 1H), 3.62 (s, 2H), 3.57-3.34 (m, 4H), 3.29-3.13 (m, 4H), 3.04-2.82 (m, 2H), 2.64-2.39 (m, 2H), 2.23-1.40 (br m, 6H). HPLC (max plot) 99% Rt 3.01 min. LC/MS: (MS+) 395.4 (M+H⁺).

### Example 30

### (±) (4-methoxy-piperidin-1-yl)-[1-(3-phenoxy-benzyl)-pyrrolidin-3-yl]-methanone

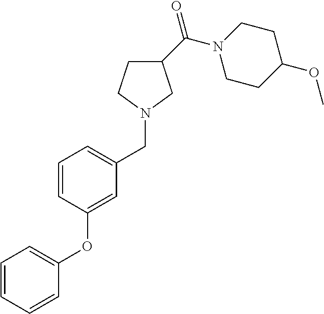 [SMILES: COC1CCN(C(=O)C2CCN(Cc3cccc(Oc4ccccc4)c3)C2)CC1]

A 2M solution of trimethylaluminum in heptane (0.64 mL; 1.28 mmol; 4 eq.) was added to a cold (0° C.) solution of 4-methoxy-piperidine hydrochloride (195 mg; 1.28 mmol; 4 eq.) in DCE (5 mL) and the resulting mixture was stirred at room temperature for 5 minutes whereupon a solution of Intermediate B3 (100 mg; 0.32 mmol; 1 eq.) in DCE (5 mL) was added. The reaction mixture was stirred at 70° C. for 48 hours. Then solution was partitioned between DCM and an aqueous solution of Rochelle's salt. The two phases were separated and the aqueous layer extracted with DCM. The combined organics were dried over sodium sulfate and concentrated in vacuo. Purification by column chromatography (5% MeOH in EA) afforded the title compound (105 mg, 83%) as colourless oil. ¹H NMR (300 MHz, DMSO-d₆) δ=7.34-7.22 (m, 3H), 7.10-6.97 (m, 5H), 6.88-6.86 (m, 1H), 3.97-3.85 (m, 1H), 3.70-3.60 (m, 3H), 3.45-3.14 (br m, 7H), 2.98-2.78 (m, 2H), 2.62-2.37 (m, 2H), 2.14-1.98 (m, 2H), 1.87-1.75 (m, 2H), 1.58-1.47 (m, 2H). HPLC (max plot) 100% Rt 3.09 min. LC/MS: (MS+) 395.4 (M+H⁺).

### Example 31

### (±) (5,7-dihydro-pyrrolo[3,4-b]pyridin-6-yl)-[3-methyl-1-(3-phenoxy-benzyl)-pyrrolidin-3-yl]-methanone

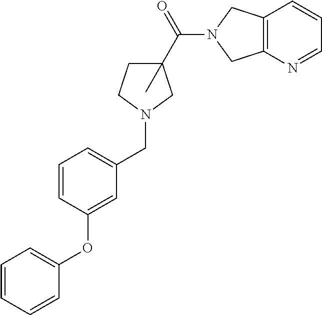

A 2M solution of trimethylaluminum in heptane (0.77 mL; 1.54 mmol; 5 eq.) was added to a cold (0° C.) solution of 6,7-Dihydro-5H-pyrrolo[3,4-b]pyridine (185 mg; 1.54 mmol; 5 eq.) in DCE (5 mL) and the resulting mixture was stirred at room temperature for 5 minutes whereupon a solution of Intermediate B2 (100 mg; 0.32 mmol; 1 eq.) in DCE (5 mL) was added. The reaction mixture was stirred at 65° C. for 24 hours. The solution was partitioned between DCM and an aqueous solution of Rochelle's salt. The two phases were separated and the aqueous layer extracted with DCM. The combined organics were dried over sodium sulfate and concentrated in vacuo. Purification by column chromatography (EA to 12% MeOH in EA) afforded the title compound 40 mg (31%) of the title compound as red oil. ¹H NMR (300 MHz, DMSO-d₆) δ=8.46 (d, J=4.9 Hz, 1H), 7.83-7.67 (m, 1H), 7.47-7.25 (m, 4H), 7.20-7.06 (m, 2H), 7.05-6.94 (m, 3H), 6.92-6.81 (m, 1H), 5.05-4.52 (m, 4H), 3.56 (s, 2H), 2.92 (d, J=9.2 Hz, 1H), 2.81-2.62 (m, 1H), 2.48-2.28 (m, 3H), 1.84-1.63 (m, 1H), 1.32 (s, 3H). HPLC (max plot) 99% Rt 2.75 min. LC/MS: (MS+) 414.4 (M+H⁺).

### Example 32

### (±) (5,8-dihydro-6H-[1,7]naphthyridin-7-yl)-[3-methyl-1-(3-phenoxy-benzyl)-pyrrolidin-3-yl]-methanone

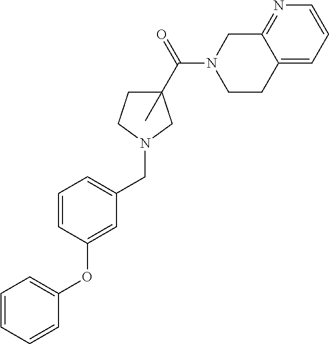

A 2M solution of trimethylaluminum in heptane (0.77 mL; 1.54 mmol; 5 eq.) was added to a cold (0° C.) solution of 5,6,7,8-tetrahydro-[1,7]naphthyridine bis hydrochloride (191 mg; 0.92 mmol; 3 eq.) in DCE (5 mL) and the resulting mixture was stirred at room temperature for 5 minutes whereupon a solution of Intermediate B2 (100 mg; 0.32 mmol; 1 eq.) in DCE (5 mL) was added. The reaction mixture was stirred at 65° C. for 24 hours. The solution was partitioned between DCM and an aqueous solution of Rochelle's salt. The two phases were separated and the aqueous layer extracted with DCM. The combined organics were dried over sodium sulfate and concentrated in vacuo. Purification by column chromatography (EA to 12% MeOH in EA) afforded the title compound (25 mg, 19%) as yellow oil. HPLC (max plot) 99% Rt 2.60 min. LC/MS: (MS+) 428.3 (M+H⁺).

### Example 33

(±) (3-methoxy-pyrrolidin-1-yl)-[1-(3-phenoxy-benzyl)-Pyrrolidin-3-yl]-methanone

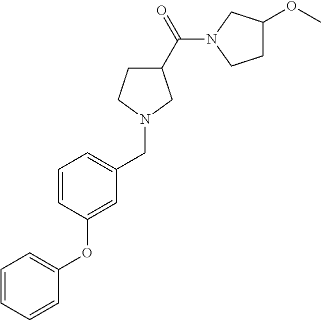 [SMILES: COC1CCN(C(=O)C2CCN(Cc3cccc(Oc4ccccc4)c3)C2)C1]

A 2M solution of trimethylaluminum in heptane (0.64 mL; 1.28 mmol; 4 eq.) was added to a cold (0° C.) solution of 3-methoxy-pyrrolidine hydrochloride (177 mg; 1.28 mmol; 4 eq.) in DCE (5 mL) and the resulting mixture was stirred at room temperature for 5 minutes whereupon a solution of Intermediate B2 (100 mg; 0.32 mmol; 1 eq.) in DCE (5 mL) was added. The reaction mixture was stirred at 70° C. for 24 hours. The solution was partitioned between DCM and an aqueous solution of Rochelle's salt. The two phases were separated and the aqueous layer extracted with DCM. The combined organics were dried over sodium sulfate and concentrated in vacuo. Purification by column chromatography (EA/MeOH/TEA, 95/5/1) afforded the title compound (85 mg, 70%) as yellow oil. ¹H NMR (300 MHz, DMSO-d₆) δ=7.29-7.17 (m, 3H), 7.05-6.91 (m, 5H), 6.85-6.80 (m, 1H), 3.95-3.84 (m, 1H), 3.62 (s, 2H), 3.56-3.32 (m, 4H), 3.25 (s, 3H), 3.10-2.80 (m, 3H), 2.51-2.31 (m, 2H), 2.10-1.86 (m, 4H). HPLC (max plot) 91% Rt 2.84 min. LC/MS: (MS+) 381.2 (M+H⁺).

### Example 34

### (±) 1-(3-phenoxy-benzyl)-pyrrolidine-3-carboxylic acid (Pyridin-2-ylmethyl)-amide

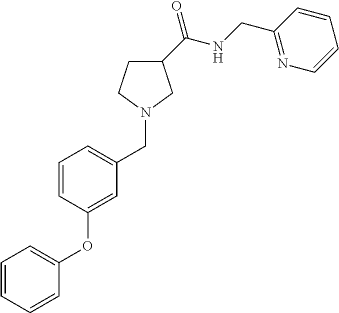 [SMILES: O=C(NCc1ccccn1)C1CCN(Cc2cccc(Oc3ccccc3)c2)C1]

A 2M solution of trimethylaluminum in heptane (1.45 mL; 2.89 mmol; 6 eq.) was added to a cold (0° C.) solution of pyridin-2-yl-methylamine (313 mg; 2.89 mmol; 6 eq.) in DCE (5 mL) and the resulting mixture was stirred at room temperature for 5 minutes whereupon a solution of Intermediate B3 (100 mg; 0.32 mmol; 1 eq.) in DCE (5 mL) was added. The reaction mixture was stirred at 70° C. for 24 hours. The solution was partitioned between DCM and an aqueous solution of Rochelle's salt. The two phases were separated and the aqueous layer extracted with DCM. The combined organics were dried over sodium sulfate and concentrated in vacuo. Purification by column chromatography (EA/MeOH/TEA, 95/5/1) afforded the title compound (42 mg, 23%) as colourless oil. ¹H NMR (300 MHz, DMSO-d₆) δ=8.59-8.49 (m, 1H), 7.86-7.60 (m, 2H), 7.37-6.86 (br m, 12H), 4.53-4.50 (m, 2H), 3.78-3.59 (m, 2H), 3.01-2.87 (m, 2H), 2.76-2.56 (m, 2H), 2.42-2.37 (m, 2H). HPLC (max plot) 92% Rt 2.37 min. LC/MS: (MS+) 388.3 (M+H⁺).

### Example 35

### Electrophysiological Assays

IonWorks electrophysiological assays were conducted to profile compounds for activity on CHO human Nav1.6 ion channels expressing cells (Milipore) using a unique protocol to assess the close (tonic, Pulse 1) and inactivated state inhibition (Pulse 2). The membrane potential is initially held at a hyperpolarized potential (−120 mV) to drive sodium channels into the resting closed state. The membrane is then reset to 0 mV voltage (for 2.5 seconds) following a brief 5 ms hyperpolarization to partially remove inactivation of non-blocked sodium channels; a short 20 ms test voltage step is applied to assess the magnitude of inhibition.

### Example A

### Injection Vials

A solution of 100 g of an active ingredient of the formula I and 5 g of diso-dium hydrogenphosphate in 3 l of bidistilled water is adjusted to pH 6.5 using 2 N hydrochloric acid, sterile filtered, transferred into injection vials, lyophilised under sterile conditions and sealed under sterile condi-tions. Each injection vial contains 5 mg of active ingredient.

### Example B

### Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### Example C

### Solution

A solution is prepared from 1 g of an active ingredient of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### Example D

### Ointment

500 mg of an active ingredient of the formula I are mixed with 99.5 g of Vaseline under aseptic conditions.

### Example E

### Tablets

A mixture of 1 kg of active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### Example F

### Coated Tablets

Tablets are pressed analogously to Example E and subsequently coated in a conventional manner with a coating of sucrose, potato starch, talc, traga-canth and dye.

### Example G

### Capsules

2 kg of active ingredient of the formula I are introduced into hard gelatine capsules in a conventional manner in such a way that each capsule con-tains 20 mg of the active ingredient.

### Example H

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1-15. (canceled)

16. Compounds of formula I, 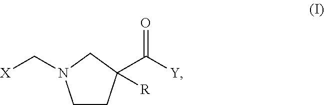
wherein:
R is H or alkyl,
X denotes one of the following groups: 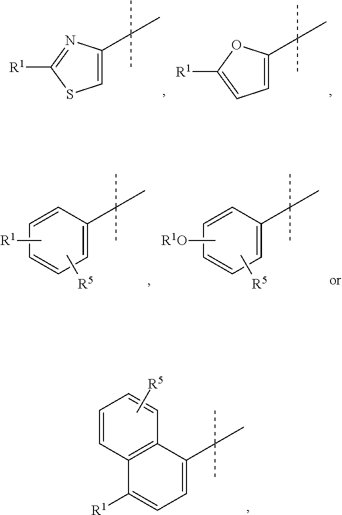
wherein:
R¹ is Ar, Het or A,
Ar denotes a monocyclic or bicyclic, unsaturated or aromatic carbocyclic ring having 6 to 14 carbon atoms which may be unsubstituted or monosubstituted, disubstituted or trisubstituted by Hal, A, CH₂OR, CH₂NR₂, OR, NR₂, NO₂, CN, COOR, CF₃, OCF₃, CONR₂, COR, phenyl and/or pyridyl,
Het denotes a monocyclic or bicyclic, saturated, unsaturated or aromatic heterocyclic ring having 1 to 3 N, O and/or S atoms which may be unsubstituted or monosubstituted, disubstituted or trisubstituted by Hal, A, CH₂OR, CH₂NR₂, OR, CF₃, OCF₃, NR₂, NO₂, CN, COOR, CONR₂, COR, phenyl and/or pyridyl,
Y is OH, Oalkyl, or NR²R³,
wherein:
R² is H or A, and
R³ is A,
A is branched or linear alkyl having 1 to 12 C-atoms, wherein one or more, H atoms may be replaced by Ar, Het, Hal, OR, CN or NR₂ and wherein one or more, CH₂-groups may be replaced by CO, phenylene, O, NR or S and/or by —CH═CH— or —C≡C— groups, or denotes cycloalkyl or cycloalkylalkylen having 3-7 ring C atoms, or
NR²R³ is selected from the following group: 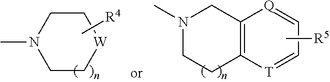
W is CHR⁶, NR⁷, or O,
R⁴ is H, OH, alkyl, or Oalkyl,
R⁵ is H, Hal, A, CH₂OR, CH₂NR₂, OR, NR₂, NO₂, CN, COOR, CF₃, OCF₃, CONR₂, COR, phenyl and/or pyridyl,
R⁶ is H or A,
R⁷ is H or alkyl,
Q, T is independently of one another N or CR⁸,
R⁸ is H or A,
n is 0, 1 or 2,
Hal denotes F, Cl, Br, I,
and pharmaceutically usable derivatives, solvates, salts and stereoisomers thereof, including mixtures thereof in all ratios.

17. Compounds of formula (I) according to claim 16 wherein X denotes one of the following groups:

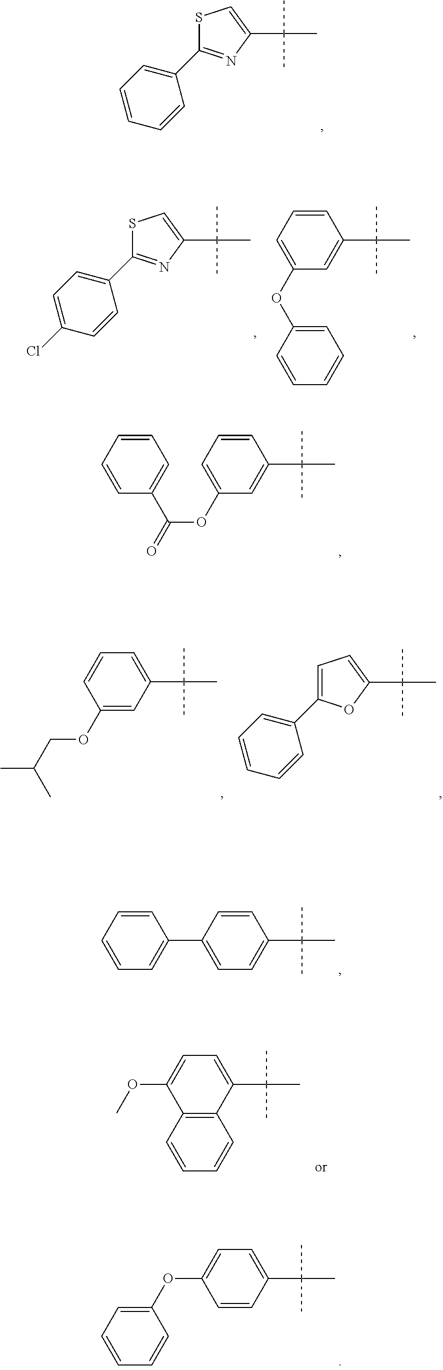

18. Compounds of formula (I) according to claim 16, wherein Y is the group —NR²R³.

19. Compounds of formula (I) according to claim 16, wherein R denotes alkyl.

20. Compounds of formula (I) according to claim 16, wherein the group —NR²R³ denotes one of the following groups:

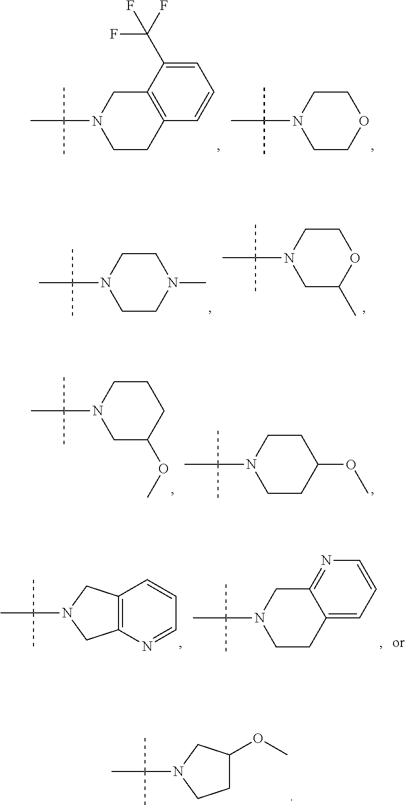

21. Compounds of formula (I) according to claim 16, wherein R² is H and R³ is alkyl, —(CH₂)₂OCH₃, or denotes one of the following groups: 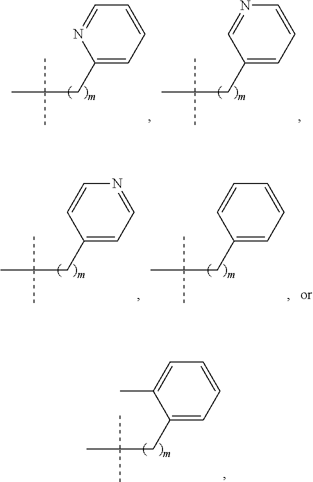
wherein m is 0, 1, 2, 3 or 2.

22. A compound selected from: 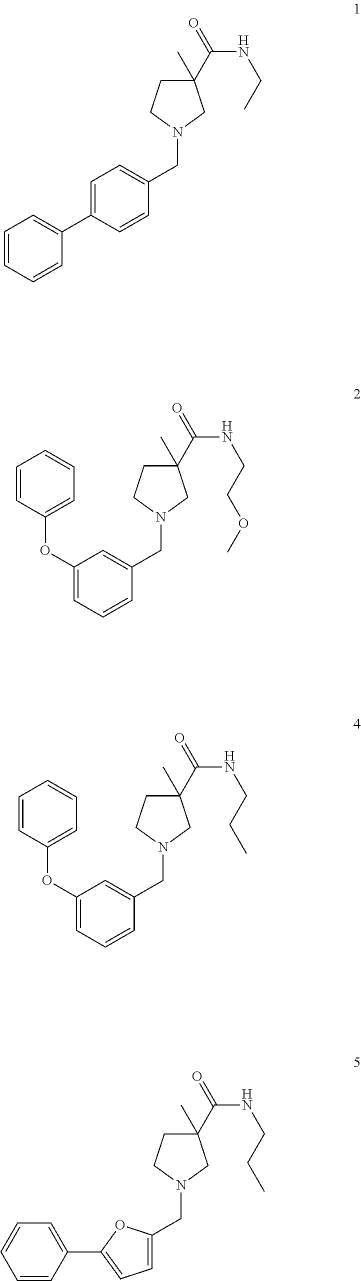 [SMILES: CCNC(=O)C1(C)CCN(Cc2ccc(-c3ccccc3)cc2)C1 ; COCCNC(=O)C1(C)CCN(Cc2cccc(Oc3ccccc3)c2)C1 ; CCCNC(=O)C1(C)CCN(Cc2cccc(Oc3ccccc3)c2)C1 ; CCCNC(=O)C1(C)CCN(Cc2ccc(-c3ccccc3)o2)C1] 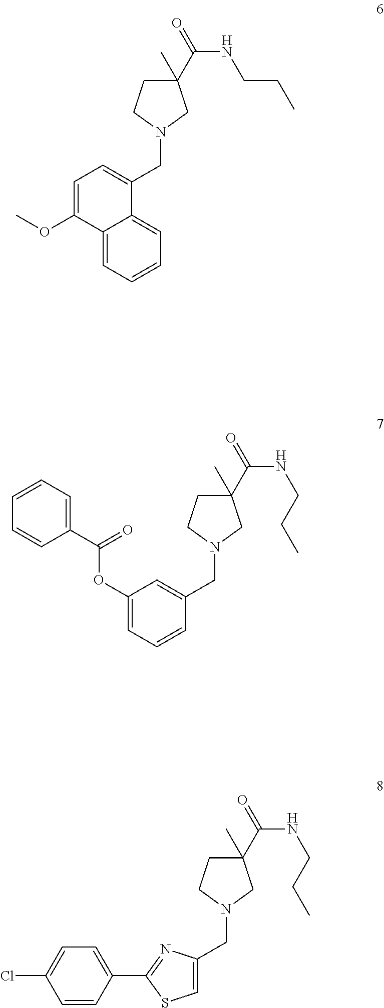 [SMILES: CCCNC(=O)C1(C)CCN(Cc2ccc(OC)c3ccccc23)C1 ; CCCNC(=O)C1(C)CCN(Cc2cccc(OC(=O)c3ccccc3)c2)C1 ; CCCNC(=O)C1(C)CCN(Cc2csc(-c3ccc(Cl)cc3)n2)C1] 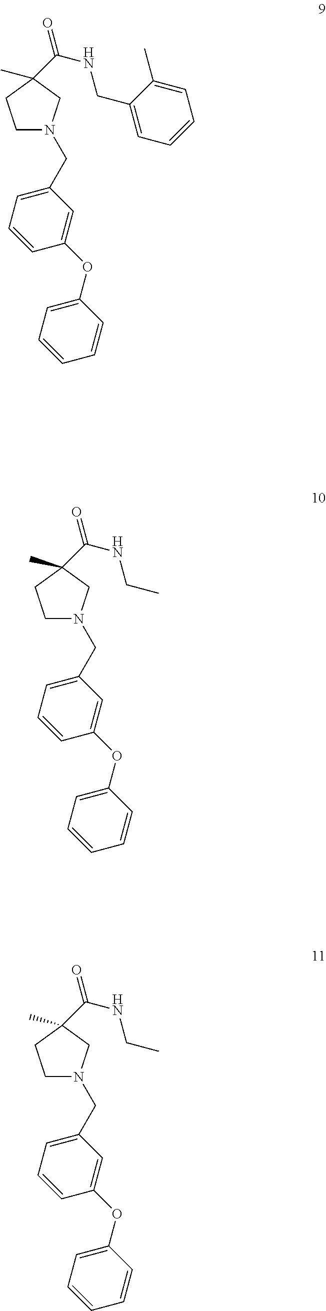 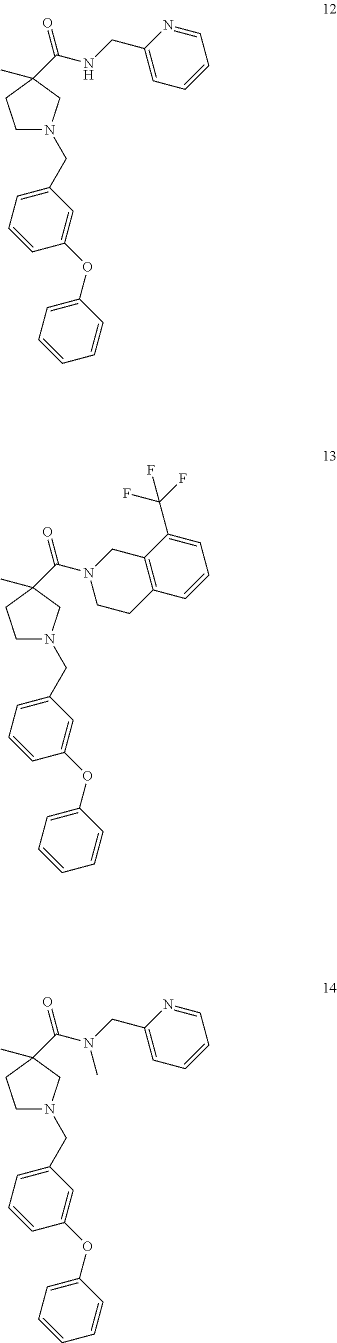 [SMILES: CC1(C(=O)NCc2ccccn2)CCN(Cc2cccc(Oc3ccccc3)c2)C1 ; CC1(C(=O)N2CCc3cccc(C(F)(F)F)c3C2)CCN(Cc2cccc(Oc3ccccc3)c2)C1 ; CN(Cc1ccccn1)C(=O)C1(C)CCN(Cc2cccc(Oc3ccccc3)c2)C1] 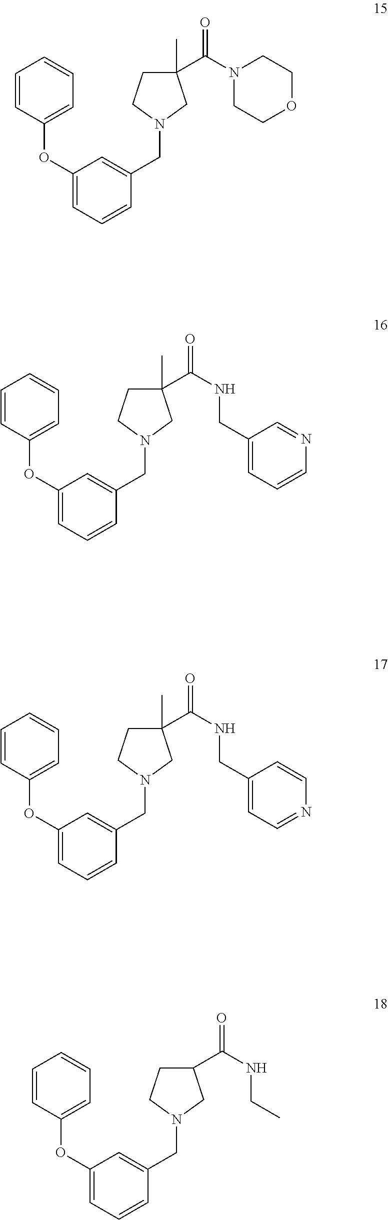 [SMILES: CC1(C(=O)N2CCOCC2)CCN(Cc2cccc(Oc3ccccc3)c2)C1 ; CC1(C(=O)NCc2cccnc2)CCN(Cc2cccc(Oc3ccccc3)c2)C1 ; CC1(C(=O)NCc2ccncc2)CCN(Cc2cccc(Oc3ccccc3)c2)C1 ; CCNC(=O)C1CCN(Cc2cccc(Oc3ccccc3)c2)C1] 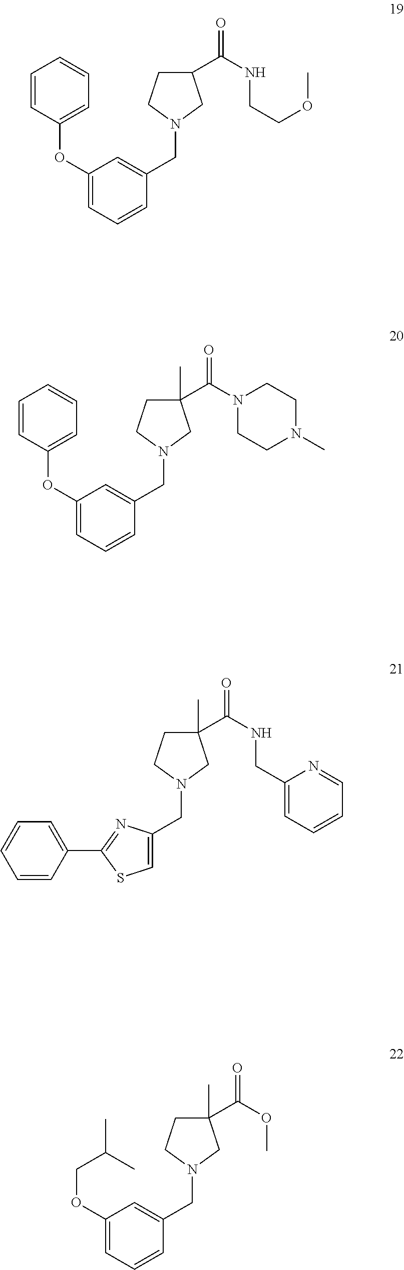 [SMILES: COCCNC(=O)C1CCN(Cc2cccc(Oc3ccccc3)c2)C1 ; CN1CCN(C(=O)C2(C)CCN(Cc3cccc(Oc4ccccc4)c3)C2)CC1 ; CC1(C(=O)NCc2ccccn2)CCN(Cc2csc(-c3ccccc3)n2)C1 ; COC(=O)C1(C)CCN(Cc2cccc(OCC(C)C)c2)C1] 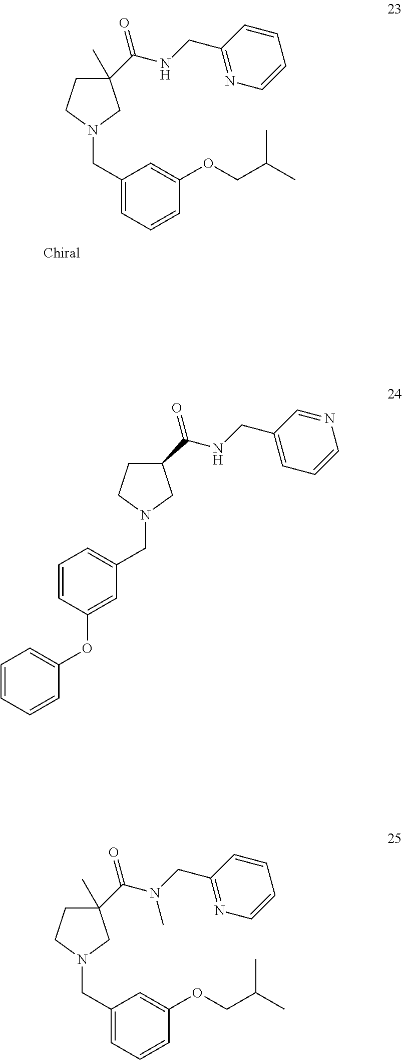 [SMILES: CC(C)COc1cccc(CN2CCC(C)(C(=O)NCc3ccccn3)C2)c1 ; O=C(NCc1cccnc1)[C@@H]1CCN(Cc2cccc(Oc3ccccc3)c2)C1 ; CC(C)COc1cccc(CN2CCC(C)(C(=O)N(C)Cc3ccccn3)C2)c1] 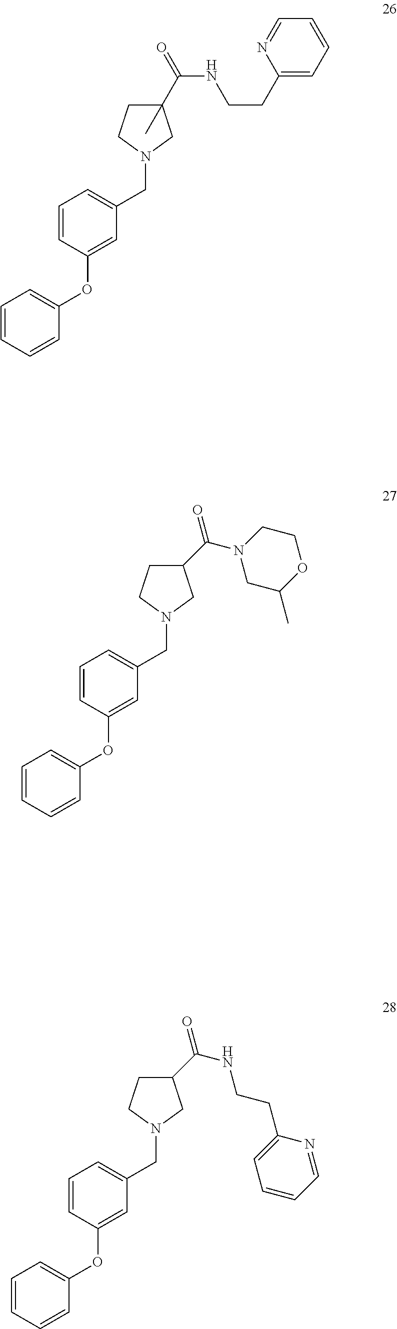 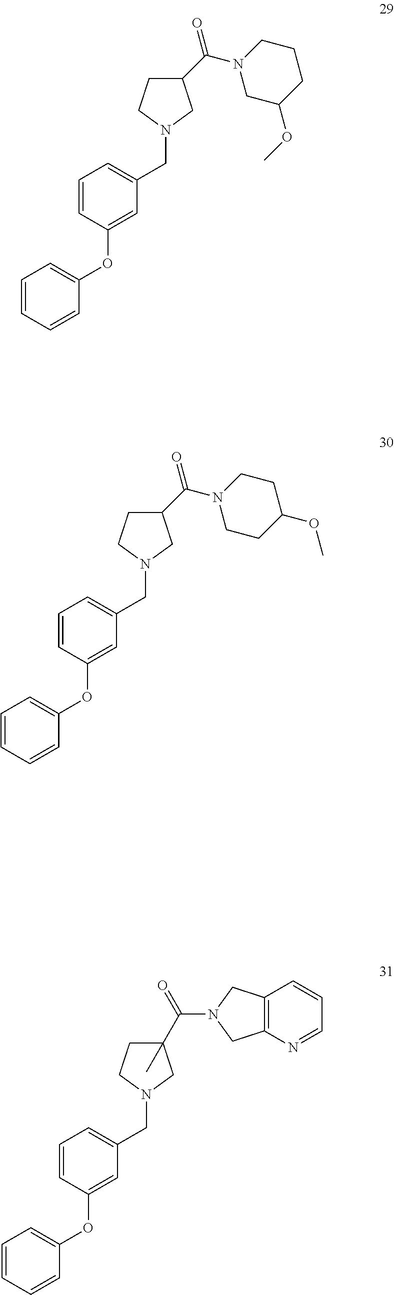 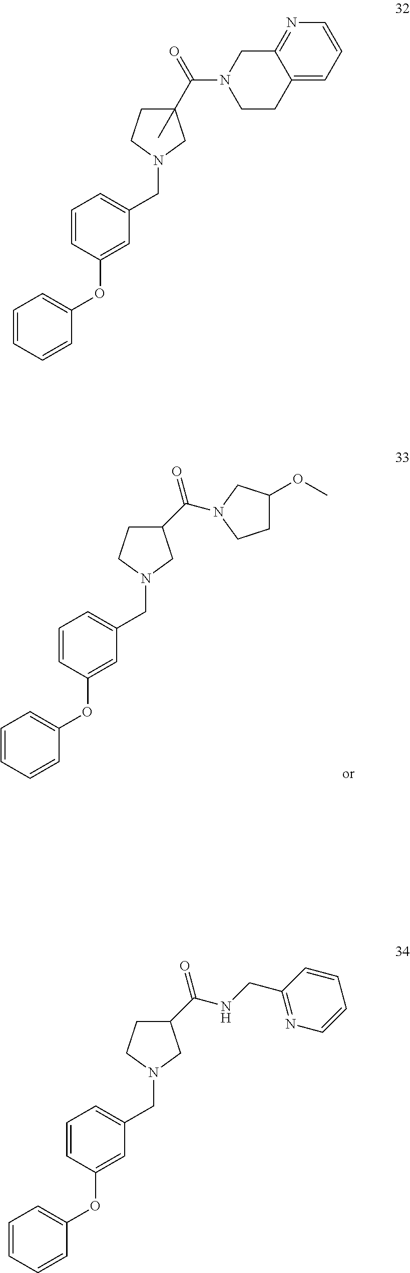
and pharmaceutically acceptable derivatives, solvates, tautomers, salts and stereoisomers thereof, including mixtures thereof in all ratios.

23. A pharmaceutical composition comprising at least one compound according to claim 16, and/or pharmaceutically usable derivatives, tautomers, salts, solvates and stereoisomers thereof, including mixtures thereof in all ratios, and optionally excipients and/or adjuvants.

24. Set (kit) consisting of separate packs of:
(a) an effective amount of a compound of formula (I) according to claim 16 and/or pharmaceutically usable derivatives, tautomers, salts, solvates and stereoisomers thereof, including mixtures thereof in all ratios,
and
(b) an effective amount of a further medicament active ingredient.

25. A method of treating an inflammatory and/or autoimmune disorder or condition comprising administering a compound according to claim 16 to a subject having an inflammatory and/or autoimmune disorder.

26. The method according to claim 25, wherein the inflammatory and/or autoimmune disorder or condition is a neurodegenerative disorder.

27. The method according to claim 26, wherein the neurodegenerative disorder is selected from multiple sclerosis, polyneuritis, multiple neuritis, amyotrophic lateral sclerosis (ALS), Alzheimer's disease and Parkinson's disease.
